SEQUENCE LISTING (6 sequences)

SEQ ID NO: 1 - DNA, 21 bp, Artificial Sequence
  Synthetic Construct
aatgatgcacatcatggtggc

SEQ ID NO: 2 - DNA, 21 bp, Artificial Sequence
  Synthetic Construct
aatgatgcacgtcatggtggc

SEQ ID NO: 3 - DNA, 41 bp, Artificial Sequence
  Synthetic Construct
tggagtatttcatgaaacaaatgaatgatgcacatcatggt

SEQ ID NO: 4 - DNA, 59 bp, Artificial Sequence
  Synthetic Construct
aaacaaatgaatgatgcacatcatggtggctggacaacaaaaatggattggatcttcca

SEQ ID NO: 5 - DNA, 69 bp, Artificial Sequence
  Synthetic Construct
tcatgaaacaaatgaatgatgcacgtcatggtggctggacaacaaaaatggattggatcttccacacaa

SEQ ID NO: 6 - DNA, 68 bp, Artificial Sequence
  Synthetic Construct
  misc_feature 29, 39: n = A,T,C or G
tatttcatgaaacaaatgaatgatgcacntcatggtggntggacaacaaaaatggattggatcttcca

(19) United States
(12) Patent Application Publication
(10) Pub. No.: US 2024/0013858 A1
(43) Pub. Date: Jan. 11, 2024

(54) METHODS FOR DETERMINING VARIANT FREQUENCY AND MONITORING DISEASE PROGRESSION

(71) Applicant: Foundation Medicine, Inc., Cambridge, MA (US)

(72) Inventors: Jonathan F. FREIDIN, Cambridge, MA (US); Mark R. KENNEDY, Cambridge, MA (US); Alyssa ANTONOPOULOS, Medford, MA (US)

(73) Assignee: Foundation Medicine, Inc., Cambridge, MA (US)

(21) Appl. No.: 18/027,826

(22) PCT Filed: Sep. 23, 2021
(86) PCT No.: PCT/US2021/051755

§ 371 Date: Mar. 22, 2023

Related U.S. Application Data
(60) Provisional application No. 63/082,939, filed on Sep. 24, 2020.

(51) Int. Cl. G16B 20/20; G16B 20/10; G16B 30/00; G16B 40/20; G16H 10/40; G16H 50/70

(52) U.S. Cl. CPC G16B 20/20; G16B 20/10; G16B 30/00; G16B 40/20; G16H 10/40; G16H 50/70; G16H 70/60

(57) ABSTRACT

Methods for determining a variant frequency in a test sample from a subject, and methods for labeling sequencing reads as having or not having a variant are described herein. Exemplary methods include generating a reference match score and a variant match score by aligning sequencing reads to a corresponding variant sequence and a corresponding reference sequence, and labeling the sequencing read as having or not having the variant based on the determined match scores. Also described herein are methods monitoring disease progression and methods of treating a subject having a disease. Further described are devices and systems for implementing such methods.

### CROSS-REFERENCE TO RELATED APPLICATION(S)

This application claims the benefit of U.S. Provisional Application No. 63/082,939, filed Sep. 24, 2020, which is hereby incorporated by reference in its entirety.

### FIELD OF THE INVENTION

Methods and systems for identifying a variant, determining a variant frequency in a test sample, methods of monitoring disease progression (such as cancer progression) and methods of treating a subject with a disease (such as cancer) are described herein.

### BACKGROUND

Genomic testing shows significant promise towards developing better understanding of cancers and managing more effective treatment approaches. Genomic testing involves the sequencing of the genome, or a portion thereof, of a patient's biological sample (which may contain cancer cells or cell-free nucleic acid products of cancer cells) and identifying any genetic variants (for example, a mutation that may be associated with a tumor) in the sample versus a reference genetic sequence. A genetic variant can include, for example, insertions, deletions, substitutions, rearrangements, or any combination thereof. Identifying and understanding these genetic variants (e.g., mutations) as they are found in a specific patient's cancer may also help develop better treatments and help identify the best approaches (or exclude ineffective approaches) for treating specific cancer variants using genomic information.

Generally, biological samples are processed in a laboratory with various possible techniques, with the end goal of extracting and isolating DNA contained therein. That isolated DNA is sequenced, resulting in a data structure representation (which may be electronic) of the DNA from the patient sample. Often, that data structure representation is in the form of several thousand “reads” or more (e.g., tens of thousands, hundreds of thousands, millions, tens of millions, or hundreds of millions reads). A single read generally comprises a relatively short (e.g., 50-150 bases) subsequence of the patient's DNA. In contrast, the entire human genome is approximately 3 billion bases long, and sub-regions of interest for the purposes of this application can be several tens of thousands bases long.

Progression of certain diseases, such as cancer, clonal hematopoiesis, can be monitored in a patient by determining variant frequency among nucleic acid molecules in a sample taken from the patient. Cancer severity is generally correlated with the number of variants within the tumor genome or the relative frequency at which those variants appear in a sample. For example, cell-free DNA is generally a mixture of genomic DNA and circulating-tumor DNA. As the severity of the cancer increases, a larger portion of the cell-free DNA is attributable to the cancer. By tracking the relative frequency of variants indicative of the tumor genome, progression of the disease can be monitored.

Variant calling pipelines generally require a threshold number of sequencing reads to be identified as having the variant before a positive variant call is made. Detecting a sufficient number of sequencing reads often requires substantial sequencing depth, which may not be possible if limited amounts of disease-associated nucleic acid is possible. There remains a need for efficient variant calling methods that have a low limit of detection and can be used for tracking disease progression.

### BRIEF SUMMARY OF THE INVENTION

Described herein is a method of labeling sequencing reads from a test sample from a subject as having or not having a genetic variant, and a method of determining a variant frequency in a test sample from a subject. Also described herein are methods of monitoring disease progression and methods of treating a subject with a disease. Further described are electronic devices and systems for carrying out such methods.

In some embodiments, the method of detecting a genetic variant or determining a variant allele frequency in a test sample from a subject comprises: (a) selecting a genetic variant at a variant locus from a variant panel; (b) obtaining one or more sequencing reads associated with the test sample that overlaps the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read, to generate labeled sequencing reads, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

The method may include sequencing nucleic acid molecules obtained from a test sample from a subject, thereby generating one or more sequencing reads.

Sequencing the nucleic acid molecules may include the use of a massively parallel sequencing (MPS) technique (e.g., next generation sequencing (NGS), whole genome sequencing (WGS), whole exome sequencing, targeted sequencing, direct sequencing, or Sanger sequencing technique.

For example, in some implementations of the method, a method of detecting a genetic variant or determining a variant allele frequency in a test sample from a subject, includes: providing a plurality of nucleic acid molecules obtained from a test sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequencing reads overlap a variant locus within a subgenomic interval in the sample; receiving, at the one or more processors, one or more sequencing reads that corresponds with a reference sequence and a variant sequence; receiving, at the one or more processors, the reference sequence from a memory; generating, at the one or more processors, a reference match score for each of the one or more sequencing reads by aligning each sequencing read to the corresponding reference sequence; receiving, at the one or more processors, the variant sequence from the memory; generating, at the one or more processors, a variant match score for each of the one or more sequencing reads by aligning each sequencing read to the corresponding variant sequence; and labeling, at the one or more processors, each of the one or more sequencing reads as either having the genetic variant, not having the genetic variant, or a being a null read, based on the reference match score and the variant match score; wherein: sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal. The one or more adapters can include amplification primers, flow cell adaptor sequences, substrate adapter sequences, or sample index sequences. The captured nucleic acid molecules may be captured from the amplified nucleic acid molecules by hybridization to one or more bait molecules. The one or more bait molecules may include one or more nucleic acid molecules, each comprising a region that is complementary to a region of a captured nucleic acid molecule. Amplifying the nucleic acid molecules may include performing a polymerase chain reaction (PCR) amplification technique, a non-PCR amplification technique, or an isothermal amplification technique.

In some embodiments, the method further comprises calling the presence of the genetic variant in the test sample based on the labeled one or more sequencing reads.

In some embodiments, the corresponding reference sequence and the corresponding variant sequence comprise the variant locus, a 5′ flanking region, and a 3′ flanking region. In some embodiments, the 5′ flanking region and the 3′ flanking region are each about 5 bases in length to about 5000 bases in length.

In some embodiments, the method further comprises generating the corresponding reference sequence or the corresponding variant sequence.

In some embodiments, the corresponding reference sequence and the corresponding variant sequence are identical except for the genetic variant.

In some embodiments, the method comprises calling the presence of the genetic variant in the test sample based on the labeled one or more sequencing reads. In some embodiments, the one or more sequencing reads comprises a plurality of sequencing reads overlapping the variant locus, and the method further comprises determining a number of sequencing reads from the plurality of sequencing reads having the genetic variant or a number of sequencing reads from the plurality of sequencing reads not having the genetic variant. In some embodiments, the method comprises determining a variant allele frequency for the genetic variant using the number of sequencing reads having the genetic variant and the number of sequencing reads not having the genetic variant.

In some embodiments, the method comprises labeling one or more sequencing reads associated with the test sample for a plurality of genetic variants at different variant loci selected from the variant panel.

In some embodiments, the method comprises determining a disease status for the subject. In some embodiments, the disease status is a value proportional to a percentage of circulating-tumor DNA (ctDNA) compared to total cell-free DNA (cfDNA) in the test sample. In some embodiments, the disease status is a maximum somatic allele fraction of cfDNA. In some embodiments, the disease status comprises a qualitative factor indicating recurrence of a cancer in the subject, the presence of a cancer resistant to a treatment modality in the subject, or the presence of a cancer that can be treated with a particular treatment modality.

In some embodiments of the methods described herein, the test sample is derived from a liquid biopsy sample from the subject. For example, the liquid biopsy sample may include blood, plasma, cerebrospinal fluid, sputum, stool, urine, or saliva. In some implementations, the liquid biopsy sample includes circulated tumor cells (CTCs). In some implementations, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the test sample comprises cfDNA. In some implementations, the test sample incudes a mixture of tumor nucleic acid molecules and non-tumor nucleic acid molecules. In some implementations, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments of the described methods, the test sample is derived from a solid tissue biopsy sample from the subject. Optionally, the method may further include obtaining the test sample from the subject.

In some embodiments, the reference match score and the variant match score are determined using a sequence alignment algorithm. In some embodiments, the sequence alignment algorithm is a Smith-Waterman alignment algorithm, a Striped Smith-Waterman alignment algorithm, or a Needleman-Wunsch alignment algorithm.

In some embodiments, the genetic variant comprises a single nucleotide variant (SNV), a multiple nucleotide variant (MNV), an indel, or a rearrangement junction. In some embodiments, the variant panel is determined by sequencing nucleic acid molecules in a previous test sample obtained from the subject, and calling one or more genetic variants. In some embodiments, the subject received an intervening treatment for a disease between the previous test sample being obtained and the test sample being obtained.

In some embodiments, the disease is cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CIVIL), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the method further comprises adjusting the treatment based on a difference between a disease status for the subject determined using the test sample and a previous disease status for the subject based on the previous test sample. Adjusting the disease therapy can include, for example, adjusting a dosage of the disease therapy or selecting a different disease therapy in response to the disease progression. The method may further include administering the adjusted disease therapy to the subject. In some implementations, the first sample is acquired from the subject before the subject has been administered a disease therapy, and the second sample is acquired from the subject after the subject has been administered the disease therapy. The disease therapy may include, for example, chemotherapy, radiation therapy, immunotherapy, a targeted therapy, or surgery.

In some implementations of the method, the detected genetic variant or determined variant allele frequency is used as a basis for enrolling the subject in a clinical trial for a selected disease treatment (e.g., an anticancer therapy).

Also described herein is a method of monitoring disease progression, comprising: sequencing nucleic acid molecules in a first test sample acquired from a subject with a disease to generate first sequencing reads; generating a personalized variant panel for the subject; sequencing nucleic acid molecules in a second test sample acquired from the subject at a later time point than the first test sample to generate second sequencing reads; and detecting, using the second sequencing reads, the genetic variant or determining, using the second sequencing read, the variant allele frequency using one of the methods described above. In some embodiments, the method comprises administering a disease therapy to the subject after the first test sample is acquired from the subject and before the second test sample is acquired from the subject. In some embodiments, the method comprises generating a first disease status based on a number of first sequencing reads having a variant entered into the variant panel; and generating a second disease status based on a number of second sequencing reads having a variant from within the variant panel. In some embodiments, the method further comprises determining disease progression by comparing the first disease status and the second disease status. In some embodiments, the method comprises administering a disease therapy to the subject after the first test sample is acquired from the subject and before the second test sample is acquired from the subject; and adjusting the disease therapy based on the determined disease progression.

Also described herein is a method of treating a subject with a disease (such as cancer), comprising: acquiring a first test sample from the subject; sequencing nucleic acid molecules in a first test sample to generate first sequencing reads; determining a first disease status using the first sequencing reads; generating a personalized variant panel for the subject; administering a disease therapy to the subject; acquiring a second test sample from the subject after the disease therapy has been administered to the subject; sequencing nucleic acid molecules in the second test sample to generate second sequencing reads; detecting, using the second sequencing reads, the genetic variant or determining, using the second sequencing read, the variant allele frequency using one of the methods described above; determining a second disease status using the labeled second sequencing reads; determining disease progression by comparing the first disease status and the second disease status; adjusting the disease therapy administered to subject based on the disease progression; and administering the adjusted disease therapy to the subject. In some embodiments, the disease is cancer.

In some embodiments of the above methods, the method comprises generating or updating a report comprising (1) identifying information for the subject, and (2) a call for the presence or absence of the genetic variant, or a call for the variant allele frequency for the genetic variant. In some embodiments, the method comprises transmitting the report to the subject or a healthcare provider for the subject. In some implementations, the report is transmitted via a computer network or a peer-to-peer connection.

Also described herein is a computer-implemented method of detecting a genetic variant or determining a variant allele frequency in a test sample from a subject, comprising, and an electronic device comprising one or more processors and a memory storing a reference sequence that does not comprise the genetic variant and a variant sequence comprising the genetic variant at a variant locus: receiving, at the one or more processors, one or more sequencing reads associated with the test sample that corresponds with the reference sequence and the variant sequence; receiving, at the one or more processors, the reference sequence from the memory; generating, at the one or more processors, a reference match score for each of the one or more sequencing reads by aligning each sequencing read to the corresponding reference sequence; receiving, at the one or more processors, the variant sequence from the memory; generating, at the one or more processors, a variant match score for each of the one or more sequencing reads by aligning each sequencing read to the corresponding variant sequence; and labeling, at the one or more processors, each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

In some embodiments of the computer-implemented method, the method comprises storing a label associated with each sequencing read in the memory.

In some embodiments of the computer-implemented method, the method comprises calling, using the one or more processors, a presence or absence of the genetic variant in the test sample based on the labeled one or more sequencing reads; and storing a call for the genetic variant in the memory.

In some embodiments of the computer-implemented method, the method comprises determining, using the one or more processors, the variant allele frequency of the genetic variant in the test sample based on the labeled one or more sequencing reads; and storing the variant allele frequency in the memory.

In some embodiments of the computer-implemented method, the corresponding reference sequence and the corresponding variant sequence comprise the variant locus, a 5′ flanking region, and a 3′ flanking region. In some embodiments, the 5′ flanking region and the 3′ flanking region are each about 5 bases in length to about 5000 bases in length.

In some embodiments of the computer-implemented method, the method comprises, using the one or more processors: selecting, using the one or more processors, the genetic variant from a variant panel stored on the memory; generating, using the one or more processors, the reference sequence or the variant sequence; and storing the reference sequence or the variant sequence in the memory.

In some embodiments of the computer-implemented method, the corresponding reference sequence and the corresponding variant sequence are identical except for the genetic variant.

In some embodiments of the computer-implemented method, the one or more sequencing reads comprises a plurality of sequencing reads overlapping the variant locus, and the method further comprises determining, using the one or more processors, a number of sequencing reads from the plurality of sequencing reads having the genetic variant or a number of sequencing reads from the plurality of sequencing reads not having the genetic variant.

In some embodiments of the computer-implemented method, the method comprises labeling, using the one or more processors, one or more sequencing reads associated with the test sample for a plurality of genetic variants at different variant loci selected from a variant panel.

In some embodiments of the computer-implemented method, the method comprises determining, using the one or more processors, a disease status for the subject. In some embodiments, the disease status is a value proportional to a percentage of circulating-tumor DNA (ctDNA) compared to total cell-free DNA (cfDNA) in the test sample. In some embodiments, the disease status is a maximum somatic allele fraction of cfDNA. In some embodiments, the disease status comprises a qualitative factor indicating recurrence of a cancer in the subject, the presence of a cancer resistant to a treatment modality in the subject, or the presence of a cancer that can be treated with a particular treatment modality.

In some embodiments of the computer-implemented method, the test sample comprises cfDNA.

In some embodiments of the computer-implemented method, the reference match score and the variant match score are determined using a sequence alignment algorithm. In some embodiments, the sequence alignment algorithm is a Smith-Waterman alignment algorithm, a Striped Smith-Waterman alignment algorithm, or a Needleman-Wunsch alignment algorithm.

In some embodiments of the computer-implemented method, the genetic variant comprises a single nucleotide variant (SNV), a multiple nucleotide variant (MNV), an indel, or a rearrangement junction.

In some embodiments of the computer-implemented method, the variant panel is determined by sequencing nucleic acid molecules in a previous test sample obtained from the subject, and calling one or more genetic variants. In some embodiments, the subject received an intervening treatment for a disease between the previous test sample being obtained and the test sample being obtained. In some embodiments, the disease is cancer.

In some embodiments of the computer-implemented method, the test sample is derived from a liquid biopsy sample from the subject. In some embodiments of the computer-implemented method, the test sample is derived from a solid tissue biopsy sample from the subject.

In some embodiments of the computer-implemented method, the method further comprises generating, using the one or more processors, a report comprising (1) identifying information for the subject, and (2) a call for the presence or absence of the genetic variant, or a call for the variant allele frequency. In some embodiments, the method comprises transmitting the report to a second electronic device. In some implementations, the report is transmitted via a computer network or a peer-to-peer connection.

In some embodiments of any of the above methods, the variant is a somatic mutation.

In some embodiments of any of the above methods, the variant is a germline mutation.

The method may further include, using the labeled one or more sequencing reads, or the detected genetic variant or determined variant allele frequency, to generate a genomic profile for the subject. The genomic profile for the subject may include results from a comprehensive genomic profiling (CGP) test, a gene expression profiling test, a cancer hotspot panel test, a DNA methylation test, a DNA fragmentation test, an RNA fragmentation test, or any combination thereof. In some implementations of the method, the method may further include selecting an anticancer agent, administering an anticancer agent, or applying an anticancer treatment to the subject based on the generated genomic profile. In some implementations of the method, genomic profile is used as a basis for enrolling the subject in a clinical trial for a selected disease treatment (e.g., an anticancer therapy).

In some implementations of the method, the method further includes selecting an anticancer therapy to administer to the subject based on the detection of the genetic variant or determined variant allele frequency. For example, the detection of the genetic variant or the determination of the allele frequency in the test sample may be used in making suggested treatment decisions for the subject. In some implementations of the method, the detected genetic variant or determined variant allele frequency is used as a basis for enrolling the subject in a clinical trial for a selected disease treatment (e.g., selected anticancer therapy). In some embodiments, the method further includes administering the selected anticancer therapy to the subject. For example, the selected anticancer therapy may include chemotherapy, radiation therapy, immunotherapy, a targeted therapy, or surgery.

Detection of the genetic variant or determined variant allele frequency may be used to diagnose or confirm a diagnosis of disease in the subject. Thus, also provided herein is a method for diagnosing a disease, which can include diagnosing the subject as having the diseased based on a detection of the genetic variant or determined variant allele frequency, wherein the genetic variant detected or the variant allele frequency determined according to any of the above methods.

Also provided herein is a method of identifying a patient as being eligible for a clinical trial for a disease treatment based on a detection of the genetic variant or determined variant allele frequency, wherein the genetic variant detected or the variant allele frequency determined according to any of the methods described above. The method may further include enrolling the patient in the clinical trial. In some implementations, the method may include administering the disease treatment to the patient.

The subject of any of the methods described herein may have cancer, may be at risk of having a cancer, may be routinely tested for cancer, or may be suspect of having a cancer. In some implementations, the cancer is a solid tumor. In other implementations, the cancer is a hematological cancer.

Also described herein is an electronic device, comprising: one or more processors; a memory; and one or more programs, wherein the one or more programs are stored in the memory and configured to be executed by the one or more processors, the one or more programs including instructions for: (a) selecting a genetic variant at a variant locus from a variant panel; (b) obtaining one or more sequencing reads associated with a test sample that overlaps the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

Further described herein is a non-transitory computer-readable storage medium storing one or more programs, the one or more programs comprising instructions, which when executed by one or more processors of an electronic device having a display, cause the electronic device to: (a) select a genetic variant at a variant locus from a variant panel; (b) obtain one or more sequencing reads associated with the test sample that overlaps the variant locus; (c) generate a reference match score for each of the one or more sequencing reads by aligning each sequencing read to a corresponding reference sequence, wherein the corresponding reference sequence does not comprise the genetic variant; (d) generate a variant match score for each of the one or more sequencing reads by aligning each sequencing read to a corresponding variant sequence, wherein the corresponding variant sequence comprises the genetic variant; and (e) label each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

### BRIEF DESCRIPTION OF THE DRAWINGS

FIG. 1 shows an exemplary embodiment of a method for labeling sequencing reads.

FIG. 2 shows an exemplary method for determining a variant frequency in a test sample from a subject.

FIG. 3 shows an exemplary method for monitoring disease progression.

FIG. 4 shows an exemplary computer-implemented method for determining a variant frequency in a test sample from a subject.

FIG. 5A shows an example of a computing device in accordance with one embodiment.

FIG. 5B shows an example of shows an example of a computing system in accordance with one embodiment.

FIG. 6A shows the variant distribution of variants in a panel for Sample 1 as further described in the examples.

FIG. 6B shows the variant distribution of variants in a panel for Sample 2 as further described in the examples.

FIG. 7A shows a plot of the number of variant reads detected using an exemplary method described herein (y-axis) against the number of variant reads detected using the standard variant calling protocol (x-axis) in log scale (left) and normalized (right) for Sample 1, as described in the examples.

FIG. 7B shows a plot of the variant locus depth at each variant locus for the sum of sequencing reads labeled as having the variant or not having the variant (i.e., excluding null reads) (y-axis) using an exemplary method described herein against the variant locus depth at each variant locus for the sum of sequencing reads from the initial pool of sequencing reads that overlap with the variant locus (x-axis) in log scale (left) and normalized (right) for Sample 1, as described in the examples.

FIG. 8A shows a plot of the number of variant reads detected using an exemplary method described herein (y-axis) against the number of variant reads detected using the standard variant calling protocol (x-axis) in log scale (left) and normalized (right) for Sample 2, as described in the examples.

FIG. 8B shows a plot of the variant locus depth at each variant locus for the sum of sequencing reads labeled as having the variant or not having the variant (i.e., excluding null reads) (y-axis) using an exemplary method described herein against the variant locus depth at each variant locus for the sum of sequencing reads from the initial pool of sequencing reads that overlap with the variant locus (x-axis) in log scale (left) and normalized (right) for Sample 2, as described in the examples.

FIG. 9A shows a plot of the number of variant reads detected using another exemplary method described herein (y-axis) against the number of variant reads detected using the standard variant calling protocol (x-axis) in log scale (left) and normalized (right) for Sample 1, as described in the examples.

FIG. 9B shows a plot of the variant locus depth at each variant locus for the sum of sequencing reads labeled as having the variant or not having the variant (i.e., excluding null reads) (y-axis) using another exemplary method described herein against the variant locus depth at each variant locus for the sum of sequencing reads from the initial pool of sequencing reads that overlap with the variant locus (x-axis) in log scale (left) and normalized (right) for Sample 1, as described in the examples.

FIG. 10A shows a plot of the number of variant reads detected using another exemplary method described herein (y-axis) against the number of variant reads detected using the standard variant calling protocol (x-axis) in log scale (left) and normalized (right) for Sample 2, as described in the examples.

FIG. 10B shows a plot of the variant locus depth at each variant locus for the sum of sequencing reads labeled as having the variant or not having the variant (i.e., excluding null reads) (y-axis) using another exemplary method described herein against the variant locus depth at each variant locus for the sum of sequencing reads from the initial pool of sequencing reads that overlap with the variant locus (x-axis) in log scale (left) and normalized (right) for Sample 2, as described in the examples.

### DETAILED DESCRIPTION OF THE INVENTION

Described herein are methods for determining a variant allele frequency, or detecting the presence or absence of a variant, in a test sample from a subject, methods for monitoring disease progression, methods detecting the presence of a tumor, methods for profiling an immune repertoire in a subject, methods for identifying a tumor clone, a viral strain, or a bacterial strain, methods for detecting clonal hematopoiesis, and methods for treating a disease that include monitoring disease progression and adjusting a treatment therapy based on the disease progression. Variant allele frequency determination or variant detection can utilize a personal variant panel established for a subject using an initial sample. The personalized variant panel includes genetic variants that are indicative of the disease. The variant panel can then be used to quickly label most sequencing reads from the subject as either having or not having the variant sequence. The labeled sequencing reads can be then used to determine a disease status based on variant frequency.

Making clinical decisions when treating a subject requires the treating physician to be confident in a diagnostic tool used to assess the subject. Sequencing nucleic acid molecules for a subject and de novo variant calling provides useful information that can be used characterize the disease. However, nucleic acid sequencing is generally subject to substantial noise due to mutations introduced during PCR amplification, errors made during nucleotide detection during sequencing, and other anomalies that may be introduced during the sequencing process. For this reason, many sequencing pipelines require a threshold number of unique sequencing reads having the same variant before the variant is confidently called. Sequencing at sufficiently high depth can overcome this hurdle, but can be expensive and may not be possible if limited tumor nucleic acids are available (for example, in the case of circulating tumor (ctDNA) shed from a small tumor clone). Further, certain bona fide variants may be detected but not positively called because the number of detected sequencing reads having the variant does not meet the call threshold. Using the methods described herein, however, sequencing reads labeled as having a variant from a predetermined variant panel lowers the limit of detection because the likelihood of a false positive variant call from an a priori panel is unlikely due to random chance.

Further, de novo variant calling is computationally expensive. The methods described herein streamline the variant calling process for generating more efficient variant calls and more efficient measurements of allele frequency of a given variant. For example, the methods described herein can be limited to the analysis of a selected number of loci.

In some embodiments, a method of detecting a genetic variant or determining a variant allele frequency in a test sample from a subject includes: (a) selecting a genetic variant at a variant locus from a variant panel; (b) obtaining one or more sequencing reads associated with the test sample that overlap the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read based on the reference match score and the variant match score; wherein: a sequencing read is labeled as having the genetic variant if the reference match score and the variant match score indicate that the sequencing read more closely matches the variant sequence than the reference sequence; a sequencing read is labeled as not having the genetic variant if the reference match score and the variant match score indicate that the sequencing read more closely matches the reference sequence than the variant sequence; and a sequencing read is labeled as a null read if the reference match score and the variant match score are equal. The labeled sequencing reads may then be used to determine a disease status for the subject.

The method of determining variant allele frequency can be used to monitor disease progression. For example, a method of monitoring disease progression can include sequencing nucleic acid molecules in a first test sample acquired from a subject with a disease to generate first sequencing reads; generating a personalized variant panel for the subject; sequencing nucleic acid molecules in a second test sample acquired from the subject at a later time point than the first test sample to generate second sequencing reads; and labeling the second sequencing reads using the method described herein. The labeled sequencing reads may then be used to determine a disease status for the subject, which can be compared to a previously determined disease status (e.g., a disease status associated with the subject at the time the first test sample was acquired from the subject) to monitor disease progression.

Disease status monitoring may further be used to treat a subject with a disease, for example by adjusting a disease therapy based on the monitored disease progression. For example, in some embodiments, a method of treating a subject with a disease may include acquiring a first test sample from the subject; sequencing nucleic acid molecules in a first test sample to generate first sequencing reads; generating a personalized variant panel for the subject; administering a disease therapy to the subject; acquiring a second test sample from the subject after the disease therapy has been administered to the subject; sequencing nucleic acid molecules in the second test sample to generate second sequencing reads; labeling the second sequencing reads using the method described herein; determining disease progression by comparing the first disease status and the second disease status; adjusting the disease therapy administered to subject based on the disease progression; and administering the adjusted disease therapy to the subject.

In some embodiments, the disease is cancer.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

The terms “individual,” “patient,” and “subject” are used synonymously, and refers to an animal, such as a human.

A “reference” sequence is any sequence that is used to compare to a test or subject sequence (e.g., a sequencing read), and may be a standardized reference sequence (e.g., a sequence from a standardized reference assembly, such as GRCh38 from the Genome Reference Consortium or an alternative reference assembly) or a personalized reference sequence (e.g., a sequence from a healthy tissue of a subject).

A “subgenomic interval” refers to a portion of genome or exome sequence. The subgenomic interval can be, for example, a single nucleotide position or more than one nucleotide position (e.g., at least 2, 5, 10, 50, 100, 150, or 250 nucleotide positions in length). Subgenomic intervals can comprise an entire gene, or a preselected portion thereof (for example, a coding region (or portions thereof), a preselected intron (or portion thereof) or exon (or portion thereof)).

The term “variant” refers to any sequence difference between a subject sequence and a reference sequence that is compared to the subject sequence. Accordingly, the term “variant” encompasses differences between a sequence from a healthy individual and a reference sequence that is used to identify a population variation, or a difference between a sequence from a diseased disuse (e.g., a tumor tissue) and a sequence from a healthy tissue (i.e., a mutation).

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Variant Panels

Certain methods described herein use a variant panel that includes one or more genetic variants of interest. The genetic variants may be, for example, variants that are associated with a particular disease (e.g., cancer or cancer clone) or disease state (e.g., metastasis). In some embodiments, the variant panel is a personalized variant panel. In some embodiments, the variant panel is a diseased patient population variant panel based on variants detected in a population of subjects having a particular disease.

The variant in the variant panel may be of any size. The variant is associated with a reference sequence and a variant sequence; therefore, as long as the targeted variant is known a priori, the reference and variant sequences can be readily constructed. The variants in the variant panel can include, for example, one or more single nucleotide variant (SNVs), one or more multiple nucleotide variants (MNVs), a rearrangement junction, and/or one or more indels. The MNV may include consecutive nucleotide variants a two or more nucleotide variants being queried using the constructed reference or variant sequence. In some embodiments, the variant panel includes one or more fusion variants or other rearrangement variants (e.g., an inversion or deletion event). The variants in the variant panel can include the locus of the variant and/or the variant relative to a reference sequence. Solely by way of example, a SNP variant can include the locus (e.g., a gene name and a base position within the gene, or a base position within a genome) and the variant (e.g., a C→G mutation).

The variant panel may include any number of variants that are associated with the disease, or example 1 or more, 2 or more, 5 or more, 10 or more, 25 or more, 50 or more, 100 or more, 500 or more, 1000 or more, 5000 or more, 10,000 or more, 20,000 or more, 50,000 or more, or 100,000 or more, or about 1 to about 10, about 10 to about 25, about 25 to about 100, about 100 to about 500, about 500 to about 1000, about 1000 to about 5000, about 5000 to about about 10,000 to about 20,000, about 20,000 to about 50,000, or about 50,000 to about 100,000.

The variant panel or subject variant may include a rearrangement junction, in some embodiments. A rearrangement variant, such as an insertion, deletion, or inversion generates can generate two rearrangement junctions (or more in complex rearrangements) relative to a reference sequence. The junction may be detected using the methods described herein, for example by using a variant sequence that includes at least one of the junctions.

In some embodiments, the variant panel is a personalized variant panel generated for a particular subject. A sample can be acquired for the subject, and nucleic acid molecules (e.g., DNA, RNA, or both) within the sample are sequenced to generate sequencing reads. In some embodiments, the RNA molecules are reverse transcribed to form corresponding cDNA molecules. Variants can then be called from the generated sequencing reads using known variant calling methods.

The sample obtained from the subject may include nucleic acid molecules derived from the diseased tissue or a mixture of nucleic acid molecules derived from diseased tissue and nucleic acid molecules derived from healthy tissue (or two separate samples may be analyzed, using a first sample using nucleic acid molecules derived from diseased tissue and a second sample derived from healthy tissue). For example, the sample may include cell-free DNA (cfDNA) that included circulating-tumor DNA (ctDNA, i.e., DNA naturally derived from a tumor tissue) and genomic cell-free DNA (i.e., cfDNA naturally derived from healthy tissue). The cfDNA can be sequenced and variants associated with the tumor called (either in reference to the genomic cell-free DNA, or in references to some other reference genome), and one or more of the called tumor variants can be included in the variant panel. In some embodiments, the sample may be derived from a tissue biopsy sample (e.g., a solid tissue sample or a hematological tissue sample) to obtain diseased tissue (e.g., a solid tumor biopsy sample or a hematological tumor biopsy sample) or healthy tissue. A nucleic acid sample can be derived from the tissue sample and can be used to generate sequencing reads.

In some embodiments, the variant panel is generated by calling variants between nucleic acid molecules obtained from a diseased tissue (e.g., a tumor tissue) and a healthy tissue. For example, the variants may be called using a matched normal, tumor sample.

In some embodiments, the variant panel is generated by calling variants between nucleic acid molecules obtained from plasma (e.g., cfDNA) and nucleic acid molecules obtained from peripheral blood mononuclear cells (PBMCs).

In some embodiments, the sample used to acquire nucleic acid molecules may be blood, serum, saliva, tissue (for example, solid or hematological tissue), cerebral spinal fluid, amniotic fluid, peritoneal fluid, interstitial fluid, or embryonic tissue. In some embodiments, the tissue is a fresh tissue (i.e., not frozen or preserved). In some embodiments, the tissue is a frozen or reserved tissue (e.g., a formaldehyde-fixed paraffin embedded (FFPE) or paraformaldehyde-fixed paraffin-embedded (PFPE) tissue).

In some embodiments, the sample used to generate a personalized variant panel is obtained from the subject prior to the start of a disease therapy. In some embodiments, the sample used to generate the personalized variant panel is obtained from the subject after the start of the disease therapy.

The personalized variant panel can be generated for the subject having the disease using a personalized reference genome or sequence (i.e., a non-diseased genomic sequence of the subject) or a standard reference genome or sequence (i.e., a reference genome or reference sequence assembled from one or more other individuals, such as a standard or publicly available reference sequence, such as the Genome Reference Consortium human genome build 37 (GRCh37), or other suitable reference genome). Differences between the nucleic acid molecules derived from the diseased tissue can be compared to the reference, and variants identified.

In some embodiments, the variants in the variant panel include one or more variants known to be associated with the particular disease (such as a particular cancer) or with a population of subjects having the particular disease (such as a particular cancer). For example, the variant panel may include one or more variants curated from literature.

Variants in the variant panel are associated with a corresponding reference sequence and a corresponding variant sequence that includes the locus of the variant with left and right flanking regions (i.e., a 5′ flanking region and a 3′ flanking region). The left and right flanking regions of the variant locus provides context for the variant, and are the same for both the corresponding reference sequence and the corresponding variant sequence. Thus, the corresponding reference sequence and the corresponding variant sequence are identical except for the variant itself. The corresponding variant sequence includes the variant, and the corresponding reference sequence does not include the variant (i.e., it includes the reference or “wild-type” sequence at the location of the variant). In some embodiments, the flanking regions each include about 5 bases or more, about 10 bases or more, about 15 bases or more, about 20 bases or more, about 25 bases or more, about 30 bases or more, about 50 bases or more, about 75 bases or more, about 100 bases or more, about 150 bases or more, about 200 bases or more, about 250 bases or more, about 300 bases or more, about 400 bases or more, or about 500 bases or more. In some embodiments, the flanking regions each include between about 5 bases and about 5000 bases, such as about 5 to about 10 bases, about 10 to about 20 bases, about 20 to about 50 bases, about 50 to about 100 bases, about 100 to about 200 bases, about 200 to about 500 bases, about 500 to about 1000 bases, about 1000 bases to about 2500 bases, or about 2500 bases to about 5000 bases. In some embodiments, the left and right flanking regions have the same number of bases, and in some embodiments, the left and right flanking regions have a different number of bases.

The corresponding reference sequence and the corresponding variant sequence can be generated, for example, using the reference sequence used to identify the variant (which may be a personalized reference sequence or a standard reference sequence). To generate the corresponding variant sequence, the variant is selected and right and left flanking sequences are added to the variant using the reference sequence. To generate the corresponding reference sequence, the reference sequence is used using the same base locations as the corresponding variant sequence. Thus, in some embodiments, the corresponding reference sequence and corresponding variant sequence are identical except for the genetic variant.

The variant panel may be a list stored in a table or file (e.g., a variant call format (VCF) file or other suitable file format), which may be stored in a non-transitory computer-readable memory and can be accessed by one or more processors for executing one or more of the methods described herein. In some embodiments, the corresponding reference sequence and the corresponding variant sequence are stored in the same table or file as the variant panel, and in some embodiments, the corresponding reference sequence and the corresponding variant sequence are stored in a different table or file as the variant panel.

The variant panel may be a variant panel associate with a disease (such as cancer) or a personalized variant panel associated with a disease (such as cancer) in a subject. Exemplary diseases include, but are not limited to, B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some embodiments, the variants in the variant panel are not associated with a disease. For example, the variant panel may be used to support a previous call or a putative call. Whole genome sequencing and other sequencing methods may results in calls being made with low certainty. The methods described herein can be used to support (either positively or negatively) certain calls to provide higher sequence confidence.

In some embodiments, the variant panel comprises one or more variants (e.g., SNP, MNP, rearrangement junction or indel) within any of the following genes: ABCB1, ABCC2, ABCC4, ABCG2, ABL1, ABL2, AKT1, AKT2, AKT3, ALK, APC, AR, ARAF, ARFRP1, ARID1A, ATM, ATR, AURKA, AURKB, BCL2, BCL2A1, BCL2L1, BCL2L2, BCL6, BRAF, BRCA1, BRCA2, C1orf144, CARD11, CBL, CCND1, CCND2, CCND3, CCNE1, CDH1, CDH2, CDH20, CDH5, CDK4, CDK6, CDK8, CDKN2A, CDKN2B, CDKN2C, CEBPA, CHEK1, CHEK2, CRKL, CRLF2, CTNNB1, CYP1B1, CYP2C19, CYP2C8, CYP2D6, CYP3A4, CYP3A5, DNMT3A, DOT1L, DPYD, EGFR, EPHA3, EPHA5, EPHA6, EPHA7, EPHB1, EPHB4, EPHB6, ERBB2, ERBB3, ERBB4, ERCC2, ERG, ESR1, ESR2, ETV1, ETV4, ETV5, ETV6, EWSR1, EZH2, FANCA, FBXW7, FCGR3A, FGFR1, FGFR2, FGFR3, FGFR4, FLT1, FLT3, FLT4, FOXP4, GATA1, GNA11, GNAQ, GNAS, GPR124, GSTP1, GUCY1A2, HOXA3, HRAS, HSP90AA1, IDH1, IDH2, IGF1R, IGF2R, IKBKE, IKZF1, INHBA, IRS2, ITPA, JAK1, JAK2, JAK3, JUN, KDR, KIT, KRAS, LRP1B, LRP2, LTK, MAN1B1, MAP2K1, MAP2K2, MAP2K4, MCL1, MDM2, MDM4, MEN1, MET, MITF, MLH1, MEL, MPL, MRE11A, MSH2, MSH6, MTHFR, MTOR, MUTYH, MYC, MYCL1, MYCN, NF1, NF2, NKX2-1, NOTCH1, NPM1, NQO1, NRAS, NRP2, NTRK1, NTRK3, PAK3, PAX5, PDGFRA, PDGFRB, PIK3CA, PIK3R1, PKHD1, PLCG1, PRKDC, PTCH1, PIEN, PTPN11, PTPRD, RAF1, RARA, RB1, RET, RICTOR, RPTOR, RUNX1, SLC19A1, SLC22A2, SLCO1B3, SMAD2, SMAD3, SMAD4, SMARCA4, SMARCB1, SMO, SOD2, SOX10, SOX2, SRC, STK11, SULT1A1, TBX22, TET2, TGFBR2, TMPRSS2, TOP1, TP53, TPMT, TSC1, TSC2, TYMS, UGT1A1, UMPS, USP9X, VHL, and WT1.

In some embodiments the variant is a mutation, for example a mutation associated with a tumor. In some embodiments, the variant is a somatic mutation. In some embodiments, the variant is a germline mutation.

### Labeling Sequencing Reads

Sequencing reads can be labeled as including a genetic variant or as not including a genetic variant (or as a “null read,” which indicates that the sequencing read cannot be labeled as having the variant or as not having the variant). Sequencing reads can be mapped to a location within a reference sequence, and the mapped location is used to select a genetic variant from the variant panel associated with the locus. Once the variant and the sequencing read are associated, the sequencing read is alleged with a reference sequence (i.e. a corresponding sequence that does not include the variant) to generate a reference match score, and a variant sequence (i.e., a corresponding sequence that includes the variant) to generate a variant match score. The sequencing read can be labeled as having the variant if the reference match score and the variant match score indicate that the sequencing read more closely matches with the variant sequence than the reference sequence, or as not having the variant if the reference match score and the variant match score indicate that the sequencing read more closely matches with the reference sequence. In some embodiments, the sequencing read is labeled as a null read if he reference match score and the variant match score are equal.

In some embodiments, a method of detecting the presence or absence of a variant or determining a variant allele frequency in a test sample from a subject, comprising (a) selecting a genetic variant at a variant locus from a variant panel; (b) obtaining one or more sequencing reads associated with the test sample that overlap the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read based on the reference match score and the variant match score; wherein: a sequencing read is labeled as having the genetic variant if the reference match score and the variant match score indicate that the sequencing read more closely matches the variant sequence than the reference sequence; a sequencing read is labeled as not having the genetic variant if the reference match score and the variant match score indicate that the sequencing read more closely matches the reference sequence than the variant sequence; and a sequencing read is labeled as a null read if the reference match score and the variant match score are equal.

Sequencing reads can be aligned to a reference sequence to determine a location of the sequencing read within a reference genome. The alignment can be used to generate a sequence alignment map file (e.g., a SAM or BAM file), which includes a mapping position for the read. The variant panel can then be accessed to select a genetic variant, and one or more sequencing reads that overlap the locus of the variant can be obtained (for example, by accessing the sequencing alignment map file). The overlap may be at one or more base positions of the variant (for example, if the variant is a multi-base variant). In some embodiments, sequencing reads that overlap the same single base (e.g., the first base) of the variant are used. A corresponding reference sequence and a corresponding variant sequence are also selected, wherein the corresponding reference sequence and the corresponding variant sequence are associated with the selected variant.

The reference match score for any given sequencing read is generated by aligning the sequencing read to the corresponding reference sequence, and the variant match score is generated by aligning the sequencing read to the corresponding variant sequence. The reference match score and the variant match score are generated using the same alignment algorithm so that the reference match score and the variant match score are comparable. The match score provides a value that indicates how closely matched the query sequence (i.e., the sequencing read) is to the corresponding variant sequence or corresponding reference sequence. Exemplary alignment algorithms include the Smith-Waterman Algorithm (SWA) (e.g., a Striped Smith-Waterman Algorithm) or the Needleman-Wunsch Algorithm (NWA). In some embodiments, the reference match score and the variant match score are generated using the Smith-Waterman Algorithm. In some embodiments, the reference match score and the variant match score are generated using the Striped Smith-Waterman Algorithm. In some embodiments, the reference match score and the variant match score are generated using the Needleman-Wunsch algorithm.

The sequencing reads are labeled by comparing the variant match score and the reference match score. For example, the sequencing read is labeled as having the genetic variant if the reference match score and the variant match score indicate that the sequencing read more closely matches the variant sequence than the reference sequence. The sequencing read is labeled as not having the genetic variant if the reference match score and the variant match score indicate that the sequencing read more closely matches the reference sequence than the variant sequence. In some instances, the reference match score and the variant match score are equal; in which case the sequencing read may be labeled as a null read. In some embodiments, a sequencing read labeled as a null read is excluded from further analysis.

The sequencing reads can be obtained by sequencing nucleic acid molecules in a test sample derived from a subject. A targeted sequencing method may be used, for example, a selective capture and/or selective amplification of targeted subgenomic regions. Nucleic acid molecules (e.g., a mixture of tumor nucleic acid molecules and non-tumor nucleic acid molecules) may be extracted from a test sample obtained from a subject. One or more adapters can be ligated to the nucleic acid molecules extracted from the sample. The adapters may include, for example, one or more of an amplification primer hybridization site, a flow cell adaptor sequence, a substrate adapter sequence, a sample index sequences, or a unique molecular identifier. The nucleic acid molecules can be amplified prior to sequencing (e.g., using a polymerase chain reaction (PCR) amplification technique, a non-PCR amplification technique, or an isothermal amplification technique). Targeted nucleic acid molecules can be captured from the amplified nucleic acid molecules (e.g., by hybridization to one or more bait molecules, where the bait molecules each comprise one or more nucleic acid molecules that each comprising a region that is complementary to a region of a captured nucleic acid molecule). The nucleic acid molecules extracted from the sample (or library proxies derived therefrom) can be sequenced using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (e.g., massively parallel) sequencer. Results of the assay may be generated, displayed, transmitted, and/or delivered as a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (e.g., massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

In some embodiments, the test sample is the same type of sample as the test sample used to determine the genetic variants in a personalized variant panel. Exemplary test samples include, but are not limited to blood, serum, saliva, tissue (for example, solid or hematological tissue), cerebral spinal fluid, amniotic fluid, peritoneal fluid, interstitial fluid, or embryonic tissue. In some embodiments, the tissue is a fresh tissue (i.e., not frozen or preserved). In some embodiments, the tissue is a frozen or reserved tissue (e.g., a formaldehyde-fixed paraffin embedded (FFPE) or paraformaldehyde-fixed paraffin-embedded (PFPE) tissue).

The subject may have cancer, be at risk of having a cancer, be routinely tested for canner, or be suspected of having a cancer. As further described herein, the results of the genetic variant detection or variant allele frequency determination method may be used to diagnose or confirm diagnosis of the cancer, or may be used to select a treatment for the cancer.

In some embodiments, the test sample is derived from a liquid biopsy sample (e.g., plasma, peripheral blood, etc.). In some embodiments, the liquid biopsy sample is blood, plasma, cerebrospinal fluid, sputum, stool, urine, or saliva. In some embodiments, the liquid biopsy sample comprises circulated tumor cells (CTCs). In some embodiments, the liquid biopsy sample comprises cell-free DNA (cfDNA), circulating tumor DNA (ctDNA), or a combination thereof. The liquid biopsy may be divided into two or more matched samples or sample components. For example, the sample may include a plasma component (which can include cfDNA) and a peripheral blood mononuclear cell (PBMC) component. The individual components may be analyzed separately to determine differences between the genetic profiles of each component. This can be used, for example, to identify somatic mutations or clonal hematopoiesis.

In some embodiments, the sample is derived from a solid tissue biopsy sample. The tissue biopsy may include cancerous cells, non-cancerous (i.e., healthy) cells, or a mixture thereof. In some embodiments, the tissue biopsy sample is a fresh tissue (i.e., not frozen or preserved). In some embodiments, the tissue is a frozen or reserved tissue (e.g., a formaldehyde-fixed paraffin embedded (FFPE) or paraformaldehyde-fixed paraffin-embedded (PFPE) tissue).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

The nucleic acid molecules in the test sample may be DNA, RNA, or a mixture thereof. In some embodiments, the RNA molecules are reverse transcribed to form corresponding cDNA molecules. The test sample obtained from the subject may include nucleic acid molecules derived from the diseased tissue or a mixture of nucleic acid molecules derived from diseased tissue and nucleic acid molecules derived from healthy tissue. For example, sample may include cell-free DNA (cfDNA) that included circulating-tumor DNA (ctDNA, i.e., DNA naturally derived from a tumor tissue) and genomic cell-free DNA (i.e., cfDNA naturally derived from healthy tissue). In some embodiments, the sample may be derived from a tissue biopsy sample (e.g., a solid tissue sample or a hematological tissue sample) to obtain diseased tissue (e.g., a solid tumor biopsy sample or a hematological tumor biopsy sample) or healthy tissue. A nucleic acid sample can be derived from the tissue sample and can be used to generate sequencing reads.

The described method for labeling sequencing reads can be repeated for any number of variants using different genetic variants at different loci selected from the genetic variant panel.

In some embodiments, the labeled sequencing reads are used to call the presence of the genetic variant in the sample from the subject. For example, if one or more sequencing reads (or one or more unique sequencing reads) are labeled as having the genetic variant, the presence of the genetic variant may be called. The threshold set for calling the presence of the genetic variant can be set as desired, depending on the desired confidence for making the call. For example, in some embodiments, the threshold for calling the presence of the genetic variant can be called as 1, 2, 3, 4, 5, 6, 7, 8, 9, 10 or more sequencing reads (or unique sequencing reads) labeled as having the genetic variant, wherein the presence of the genetic variant is called if the number of sequencing reads (or unique sequencing reads) labeled as having the genetic variant meets or is higher than the threshold.

In some embodiments, the labeled sequencing reads are used to determine the variant allele frequency for the variant in the sample. A variant allele frequency (Fᵢ) at locus i for the test sample can be determined using the number of sequencing reads labeled as having the variant (Vᵢ) and the number of sequencing reads as not having the variant (Rᵢ) according to

$${F}_{i} = \frac{{V}_{i}}{{V}_{i} + {R}_{i}} .$$

The methods described herein may be used to determine the variant allele frequency in a sample, two or more different tissues or samples, or two or more different components of the same sample. For example, a blood draw may be divided into plasma (which contains cfDNA) and peripheral blood mononuclear cells (PBMCs). A first variant allele frequency may be determined for the first sample or the first sample component (e.g., the plasma), and a second variant allele frequency may be determined for the second sample or second sample component (e.g., the PBMCs). The difference in variant allele frequency between, for example, nucleic acid molecules from plasma and nucleic acid molecules from PBMC is useful for subjects with clonal hematopoiesis or clonal hematopoiesis of indeterminate potential (CHIP).

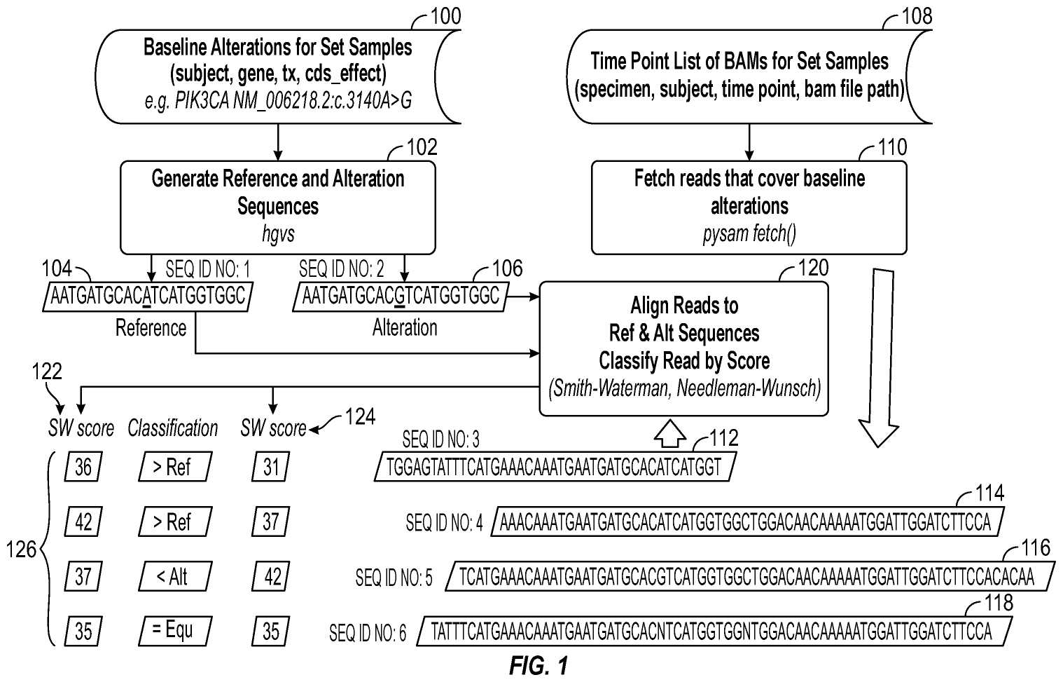

FIG. 1 shows an exemplary embodiment of a method for labeling sequencing reads. At step 100, the genetic variant panel (i.e., the baseline alternations) is generated by sequencing an initial sample obtained from the subject. The genetic variant panel may include information about each genetic variant in the panel, for example a subject identifier, the gene containing the variant, the locus of the variant, and/or the variant change (relative to reference). At corresponding sequence generation module 102, corresponding reference sequence 104 and corresponding variant sequence read 106 are generated using a variant from the variant panel and a reference sequence used to provide context for the variant. The corresponding reference sequence 104 and the corresponding variant sequence read 106 are identical except for at the variant locus, wherein an A→G SNP is present (indicated by underline). Sequencing reads obtained by sequencing a second test sample acquired from a subject are aligned to a reference sequence, and the mapped sequencing reads are included in an alignment map file 108. The alignment map file 108 includes the sequences from the sequencing reads, along with the locus information for the sequencing reads. Optionally, the alignment map file 108 may include additional information, such as information about the subject, the time point at which the sample was acquired, and/or other sample information. A variant is selected from the variant table, and sequencing reads that overlap the locus of the variant read are retrieved from the alignment map file 108 at sequencing read retrieving module 110. In the example shown in FIG. 1, sequencing reads 112, 114, 116, and 118 represent the sequencing reads that overlap the locus of the selected variant. At alignment module 120, the sequencing reads 112, 114, 116, and 118 are each aligned with the corresponding reference sequence 104 to generate a reference match score 122, and the corresponding variant sequence read 106 to generate a variant match score 124. The reference match score 122 and the variant match score 124 can be generated using an alignment algorithm, such as a Smith-Waterman algorithm or a Needleman-Wunsch algorithm. At classification module 126, for each sequencing read, the reference match score and the variant match score are compared to label the sequencing read as a having the variant, not having the variant, or being a null read. In the example illustrated in FIG. 1, sequencing reads 112 and 114 are labeled not having the variant because the reference match score is greater than the variant match score for each read (i.e., the sequencing read more closely matches the corresponding reference sequence than the corresponding variant sequence). Sequencing read 116 is labeled as having the variant because the variant match score is greater than the reference match score (i.e., the sequencing read more closely matches the corresponding variant sequence than the corresponding reference sequence). Sequencing read 118 is labeled as a null read because the variant match score equals the reference match score.

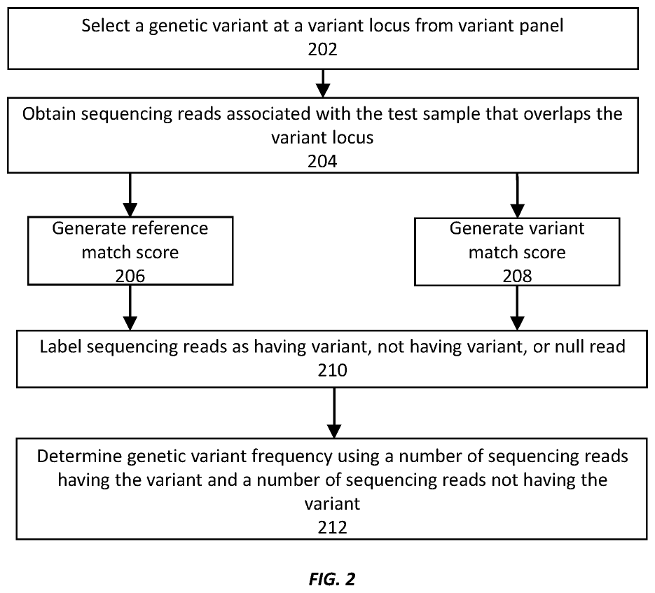

FIG. 2 shows an exemplary method for determining a variant frequency in a test sample from a subject. At step 202, a genetic variant at a variant locus is selected from a variant panel. In some embodiments, the variant panel is a personalized variant panel. At step 204, sequencing reads that overlap the variant locus and are associated with the test sample are obtained. A reference match score for each sequencing read is obtained by aligning the sequencing reads to a corresponding reference sequence at step 206, and a variant match score for each sequencing read is generated by aligning the sequencing reads to a corresponding variant sequence at step 208. Using the reference match score and the variant match score, the sequencing reads are labeled as having the variant, not having the variant, or as a null read at step 210. At step 212, the genetic variant frequency is determined using the number of sequencing reads labeled as having the variant and the number sequencing reads labeled as not having the variant.

In some embodiments, the method includes generating or updating a report (such as a printed report or an electronic medical record). The report can include one or more of a call for the presence or absence of the genetic variant, a call for the variant allele frequency, and/or a disease status. The report can also include identifying information for the subject (e.g., name, identification number, etc.). The report may be stored or transmitted to another person or entity, for example, the subject or a healthcare provider (e.g., a doctor, nurse, caretaker, hospital, clinic, etc.).

### Disease Status and Monitoring Disease Progression or Recurrence

A disease status can be determined using the variant frequency in the test sample at one or more variant loci. In some embodiments, an increase in variant frequency indicates an increase in the severity of the disease. In some embodiments, sequencing reads labeled as having the genetic variant are attributed to disease tissue. In some embodiments, sequencing reads labeled as not having the genetic variant are attributed to the non-diseased tissue. In some embodiments, sequencing reads labeled as having the genetic variant are attributed to disease tissue, and sequencing reads labeled as not having the genetic variant are attributed to the non-diseased tissue. In some embodiments, sequencing reads labeled as having the genetic variant are attributed to a first diseased tissue, and sequencing reads labeled as not having the genetic variant are attributed to a second diseased tissue and/or a non-diseased tissue.

In some embodiments, one or more genetic variants are used to characterize the disease or cancer. For example, the presence of one or more genetic variants may be used to trace the original source of the disease (e.g., a primary cancer). In some embodiments, the detection of one or more genetic variants can be used to characterize a therapy-resistant cancer or cancer as being particularly susceptible to a particular treatment. A variant panel used to characterize the disease may be based on known variants, for example those curated from literature.

In some embodiments, the disease status is determined on a per variant status. In some embodiments, the disease status is determined using a plurality of variants from the variant panel. For example, in some embodiments, a disease status (DS) can be determined using a total number of sequencing reads (or a total number of unique sequencing reads) determined as having a variant (V_{T}) and a total number of sequencing reads (or a total number of unique sequencing reads) determined as not having a variant (R_{T}), according to

$$DS = \frac{{V}_{T}}{{V}_{T} + {R}_{T}} .$$

The disease status may be determined for a plurality of genetic variants, for example as a summary statistic. In some embodiments, variants associated with germline mutations are excluded from the determination of the disease status. In some embodiments, variants associated with clonal hematopoiesis are excluded from determination of the disease status. In some embodiments, the disease status is qualitatively assessed, for example by identifying the subject has having cancer, having a recurrence of the cancer, having a cancer that is resistant to a particular treatment modality, or having a cancer that can be treated with a particular treatment modality. In some embodiments, the disease status is quantitatively assessed (e.g., a determined tumor fraction of cfDNA, or a maximum somatic allele fraction of cfDNA).

Disease progression can be monitored by determining a disease status at two or more time points. The disease status can be indicated by the variant frequency in the test sample. For example, a first test sample may be obtained from the subject at a first time point, and a second test sample may be obtained from the subject at a second time point. In some embodiments, the first test sample is used to generate the variant panel and is used to determine the disease status at the first time point, and the second test sample uses the generated variant panel to determine the disease status at the second time point.

The subject may receive treatment for the disease between the first test sample and the second test sample (i.e., an intervening treatment). Thus, by monitoring the disease progression, it can be determined whether the treatment therapy is effective in treating the disease. The treatment therapy may further be adjusted depending on the disease progression. For example, a therapeutic dose may be increased or an alternative treatment therapy used if the disease worsens or fails to improve.

The time period between the first time point and the second time point can be as frequent as desired to effectively monitor the subject. In some embodiments, the first time point and the second time point is about 1 week or more, about 2 weeks or more, about 4 weeks or more, about 8 weeks or more, about 12 weeks or more, about 16 weeks or more, about 6 months or more, about 1 year or more, or about 2 years or more.

In some embodiments, monitoring the subject for disease progression includes monitoring the subject for disease recurrence. For example, a subject deemed to be in remission may have a minimal amount of residual disease that has some recurrence risk. A test sample of the subject may be occasionally obtained and a disease status determined to see if the disease has recurred. If the disease status has recurred, then the subject can be treated for the recurring disease.

In some embodiments, a method of monitoring disease progression includes sequencing nucleic acid molecules in a first test sample acquired from a subject with a disease to generate first sequencing reads; generating a personalized variant panel for the subject; sequencing nucleic acid molecules in a second test sample acquired from the subject at a later time point than the first test sample to generate second sequencing reads; and labeling the second sequencing reads. The sequencing reads may be labeled, for example, by selecting a genetic variant at a variant locus from the personalized variant panel; (b) obtaining one or more sequencing reads associated with the test sample that overlap the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

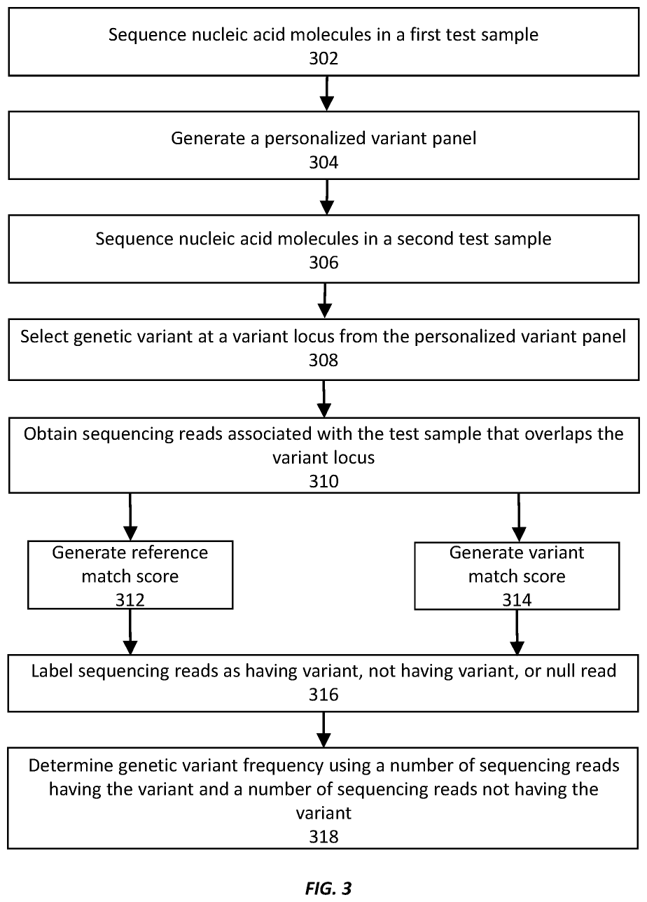

FIG. 3 shows an exemplary method for monitoring disease progression. The method includes, at step 302, sequencing nucleic acid molecules in a first test sample obtained from a subject with a disease to generate first sequencing reads. From the first sequencing reads, a personalized variant panel is generated for the subject. Optionally, a disease status for the subject can be determined, which is indicative of the disease severity for the subject. The disease status may be represented, for example, by a variant frequency determined for the subject. After a period of time, a second test sample can be obtained from the subject. At step 306, nucleic acid molecules in the second test sample are sequenced. A step 308, a genetic variant at a variant locus is selected from the personalized variant panel. At step 310, sequencing reads that overlap the variant locus and are associated with the test sample are obtained. A reference match score for each sequencing read is obtained by aligning the sequencing reads to a corresponding reference sequence at step 312, and a variant match score for each sequencing read is generated by aligning the sequencing reads to a corresponding variant sequence at step 314. Using the reference match score and the variant match score, the sequencing reads are labeled as having the variant, not having the variant, or as a null read at step 316. At step 318, the genetic variant frequency is determined using the number of sequencing reads labeled as having the variant and the number sequencing reads labeled as not having the variant. Using the determined variant frequency, a disease status for the subject can be determined indicating the severity of the disease that the time the second sample is obtained from the subject.

In some embodiments, the monitored disease is a cancer. For example, in some embodiments, the disease is B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CIVIL), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CIVIL), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the methods described herein are used to identify a viral or bacterial strain. Bacteria and viruses can mutate, and clearly distinguishing between particular strain types can be particularly important for treating an infected subject. For example, it is important to know whether a strain of Staphylococcus aureus infecting a subject is resistant to methicillin and/or vancomycin. Antibiotic or other drug resistant bacteria and viruses have a genomic signature, and the methods described herein can be used to quickly characterize different strains.

In some instances, the disclosed methods for detecting a genetic variant or determining a variant allele frequency in a test sample from a subject may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for detecting a genetic variant or determining a variant allele frequency as part of a genomic profiling process can improve the validity of, e.g., disease detection calls, made on the basis of the genomic profile by, for example, independently confirming the presence of a disease or cancer driver mechanism (e.g., an impaired DNA mismatch repair (MMR) mechanism) in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

The genomic profile may be used to select an anticancer agent, administer an anticancer agent, or apply an anticancer treatment to the subject (i.e., a decision about the selection, administration, or application of the anticancer treatment may be based on the generated genomic profile). In some implementations of the method, genomic profile is used as a basis for enrolling the subject in a clinical trial for a selected disease treatment (e.g., an anticancer therapy).

### Disease Treatments and Detection Assays

The methods described herein may be used when treating a subject with a disease. For example, the detection of the genetic variant or the determination of the allele frequency in the test sample may be used in making a treatment (e.g. a cancer treatment) decision or suggesting a treatment decision for the subject. In another example, the detection of the genetic variant or the determination of the allele frequency in the test sample may be used in adjusting a disease (e.g., cancer) therapy. As discussed above, the method may include monitoring disease progression, such as cancer progression in the subject. Monitoring disease progression allows a clinician to provide better treatment decisions, and can be used to screen for disease (e.g., cancer) recurrence or metastasis.

A first test sample can be acquired from a subject having the disease, and nucleic acid molecules from the test sample can be sequenced to generate first sequencing reads, which are used to generate a personalized variant panel for the subject. A disease therapy is then administered to the subject and, after a period of time, a second test sample is acquired from the subject at a second time point. Nucleic acid molecules from the second test sample can be sequence to generate second sequencing reads, and the second sequencing reads can be labeled using the methods described herein. For example, the second sequencing reads may be labeled by selecting a genetic variant at a variant locus from the personalized variant panel; (b) obtaining one or more sequencing reads associated with the test sample that overlap the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal. A first disease status can be determined using the first sequencing reads, and a second disease status can be determined using the labeled second sequencing reads. Disease progression can be determined by comparing the first disease status and the second disease status. The disease therapy administered to the subject can be adjusted based on the disease progression, and the adjusted disease therapy can then be administered to the subject.

A detected genetic variant or a determined variant allele frequency may be used as a basis to adjust a dosage of a disease therapy (e.g., anticancer therapy) or select a different disease therapy in response to a disease progression. The adjusted disease therapy may then be administered to the subject.

In some implementations of the method, the detected genetic variant or determined variant allele frequency is used as a basis for enrolling the subject in a clinical trial for a selected disease treatment (e.g., an anticancer therapy). For example, the clinical trial may enroll patients that have (or do not have) one or more predetermined genetic variants, and may be treated in the clinical trial with a selected disease treatment (e.g., an anticancer therapy).

In an exemplary embodiments, a method of treating a subject with a disease (such as cancer) includes: acquiring a first test sample from the subject; sequencing nucleic acid molecules in a first test sample to generate first sequencing reads; determining a first disease status using the first sequencing reads; generating a personalized variant panel for the subject; administering a disease therapy to the subject; acquiring a second test sample from the subject after the disease therapy has been administered to the subject; sequencing nucleic acid molecules in the second test sample to generate second sequencing reads; labeling the second sequencing reads by (a) selecting a genetic variant at a variant locus from a variant panel; (b) obtaining one or more sequencing reads associated with the test sample that overlaps the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal; determining a second disease status using the labeled second sequencing reads; determining disease progression by comparing the first disease status and the second disease status; adjusting the disease therapy administered to subject based on the disease progression; and administering the adjusted disease therapy to the subject.

In some embodiments, the disease therapy (such as anticancer therapy for treating a cancer) comprises surgery (for example, an excision surgery to remove one or more cancers). In some embodiments, the disease therapy comprises a radiation therapy (such as external beam radiation therapy, stereotactic radiation, intensity-modulated radiation therapy, volumetric modulated arc therapy, particle therapy (such as proton therapy), auger therapy, brachytherapy, or systemic radioisotope therapy). In some embodiments, the disease therapy comprises the administration of one or more chemical agents (for example, an anticancer agent), such as one or more chemotherapeutic agents for the treatment of cancer. Exemplary chemotherapeutic agents include, but are not limited to, anthracyclines (such as daunorubicin, epirubicin, idarubicin, mitoxantrone, valrubicin) alkylating or alkylating-like agents (such as carboplatin, carmustine, cisplatin, cyclophosphamide, melphalan, procarbazine, or thiotepa), or taxanes (such as paclitaxel, docetaxel, or taxotere). In some instances, the method can further include administering an anticancer agent or applying an anticancer treatment to the subject based on the generated genomic profile. An anticancer agent or anticancer treatment can refer to a compound that is effective in the treatment of cancer cells. Examples of anticancer agents or anticancer therapies include, but not limited to, alkylating agents, antimetabolites, natural products, hormones, chemotherapy, radiation therapy, immunotherapy, surgery, or a therapy configured to target a defect in a specific cell-signaling pathway, e.g., a defect in a DNA mismatch repair (MMR) pathway.

In some embodiments, the therapy is an immunotherapy. In some embodiments, the therapy is an immune checkpoint inhibitor.

In some embodiment, the disease therapy is a targeted therapy. Exemplary targeted therapies include tyrosine-kinase inhibitors (e.g., imatinib, gefitinib, erlotinib, sorafenib, sunitnib, dasatinib, lapatinib, nilotinib, bortezomib, JAK inibitors (e.g., tofacitinib), ALK inibitors (e.g., crizotinib), BCL-2 inhibitors (e.g., obatoclax, navitoclax, gossypol), PARP inibitiors (e.g., iniparib, olaparib), PI3K inibhtors (e.g., perifosine), apatinib, BRAF inhibitors (e.g., vemurafenib, dabrafenib, LGX818), MEK inhibitors (e.g., trametinib, MEK162), CDK inhibitors, Hsp90 inhibitors, or salinomycin), serine/threonine kinase inhibitors (e.g., temsirolimus, everolimus, vemurafenib, trametinib, or dabrafenib), or a monocolonal antibody (e.g., pembrolizumab, rituximab, trastuzumab, alemtuzumab, cetuximab, panitumumab, or bevacizumab).

In some embodiments, the therapeutic agent or anticancer therapy administered to the subject is selected based on (e.g., responsive to) calling a genetic variant in the sample using the methods described herein. The selected anticancer therapy may be administered to the subject. Exemplary selected cancer therapies may be chemotherapy, radiation therapy, immunotherapy, a targeted therapy, or surgery. For example, the detection of specific biomarkers using the methods described herein can be used as a basis for selecting a particular therapy modality. The selected anticancer therapy may be administered to the subject. Exemplary selected cancer therapies may be chemotherapy, radiation therapy, immunotherapy, a targeted therapy, or surgery. Exemplary personalized therapy selections for a given identified mutations are listed in Table 1.

TABLE 1

| Tumor Type | Genetic Variant Call | Therapy |
|---|---|---|
| Non-small | EGFR exon 19 deletions or | Afatinib, |
| cell lung cancer | EGFR exon 21 L858R | gefitinib, |
| (NSCLC) | alterations | osimertinib, |
| | | or erlotinib |
| | EGFR exon 20 T790M | Osimertinib |
| | alterations | |
| | ALK rearrangement | Alectinib, |
| | | crizotinib, |
| | | or ceritinib |
| | BRAF V600E | Dabrafenib and |
| | | trametinib |
| | | combination |
| | MET single nucleotide | Capmatinib |
| | variant or indel | |
| | that results in | |
| | MET exon 14 skipping | |
| Melanoma | BRAF V600E | Dabrafenib, |
| | | or vemurabenib |
| | BRAF V600E and V600K | Trametinib; or |
| | | cobimetinib in |
| | | combination with |
| | | vemurafenib |
| Breast Cancer | PIK3CA C420R, E542K, | Alpelisib |
| | E545A, E545D (1635G > T), | |
| | E545G, E545K, Q546, | |
| | Q546R, H1047L, H1047R, | |
| | or H1047 | |
| Colorectal cancer | KRAS wild-type (i.e., | Cetuxmab |
| | absence of variant) in | |
| | codon 12 and 13 | |
| | KRAS wild-type (i.e., | Panitumumab |
| | absence of variant) in | |
| | codon 2, 3, and 4; and/or NRAS | |
| | wild type (i.e., absence of | |
| | variant) in codon 2, 3, and 4 | |
| Ovarian cancer | BRCA½ variant | Olaparib or |
| | | rucaparib |
| Cholangiocarcinoma | FGFR2 fusion or | Pemigatinib |
| | rearrangement | |
| Prostate cancer | Variant in a Homologous | Olaparib |
| | Recombination Repair (HRR) | |
| | gene variant (e.g., one or | |
| | more of BRCA1, BRCA2, | |
| | ATM, BARD1, BRIP1, | |
| | CDK12, CHEK1, CHEK2, | |
| | FANCL, PALB2, RAD51B, | |
| | RAD51C, RAD51D, or | |
| | RAD54L) | |

In some embodiments, the treated disease is a cancer. For example, in some embodiments, the disease is B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CIVIL), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Detection of the genetic variant or determined variant allele frequency may be used to diagnose or confirm a diagnosis of disease (such as cancer) in the subject. For example, one or more genetic variants may be associated with a disease (e.g., cancer or a particular cancer type), and a diagnosis may be made on such an association.

Detection of the genetic variant or determined variant allele frequency may be used to identify a patient as being eligible for a clinical trial for a disease treatment (e.g., an anticancer treatment for a patient having cancer). Once identified, the patient may be enrolled in the clinical trial. The method may further include administering the disease treatment to the patient.

### Computer Systems and Methods

The methods described herein may be implemented using one or more computer systems. Such computer systems can include one or more programs configured to execute one or more processors for the computer system to perform such methods. One or more steps of the computer-implemented methods may be performed automatically.

In some embodiments, the computer-implemented method for detecting the presence of a genetic variant and/or determining a variant allele frequency in a test sample from a subject, or labeling sequencing reads associated with a test sample from a subject, includes (a) selecting, using one or more processors, a genetic variant at a variant locus from a variant panel stored in a memory; (b) receiving, at the one or more processors, one or more sequencing reads stored in the memory, wherein the sequencing reads are associated with the test sample that overlaps the variant locus; (c) generating, using the one or more processors, a reference match score for each of the one or more sequencing reads by aligning each sequencing read to a corresponding reference sequence retrieved from the memory, wherein the corresponding reference sequence does not comprise the genetic variant; (d) generating, using the one or more processors, a variant match score for each of the one or more sequencing reads by aligning each sequencing read to a corresponding variant sequence retrieved from the memory, wherein the corresponding variant sequence comprises the genetic variant; and (e) labeling, using the one or more processors, each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

In some embodiments of the computer-implemented method, the method further includes generating the corresponding reference sequence and/or the corresponding variant sequence. In some embodiments, the corresponding reference sequence and the corresponding variant sequence are identical except for the genetic variant.

In some embodiments of the computer-implemented method, the one or more sequencing reads comprises a plurality of sequencing reads overlapping the variant locus, and the method further comprises determining a number of sequencing reads from the plurality of sequencing reads having the genetic variant or a number of sequencing reads from the plurality of sequencing reads not having the genetic variant. In some embodiments, the method further comprises determining a variant frequency for the genetic variant using the number of sequencing reads having the genetic variant and the number of sequencing reads not having the genetic variant.

In some embodiments of the computer-implemented method, the method includes labeling one or more sequencing reads associated with the test sample for a plurality of genetic variants at different variant loci selected from the variant panel.

In some embodiments of the computer-implemented method, the method includes determining a disease status for the subject. For example, the disease status may be a value proportional to a percentage of circulating-tumor DNA (ctDNA) compared to total cell-free DNA (cfDNA) in the test sample.

In some embodiments, the reference match score and the variant match score are determined using a sequence alignment algorithm. In some embodiments, the reference match score and the variant match score are determined using a Smith-Waterman alignment algorithm. In some embodiments, the reference match score and the variant match score are determined using a Needleman-Wunsch alignment algorithm.

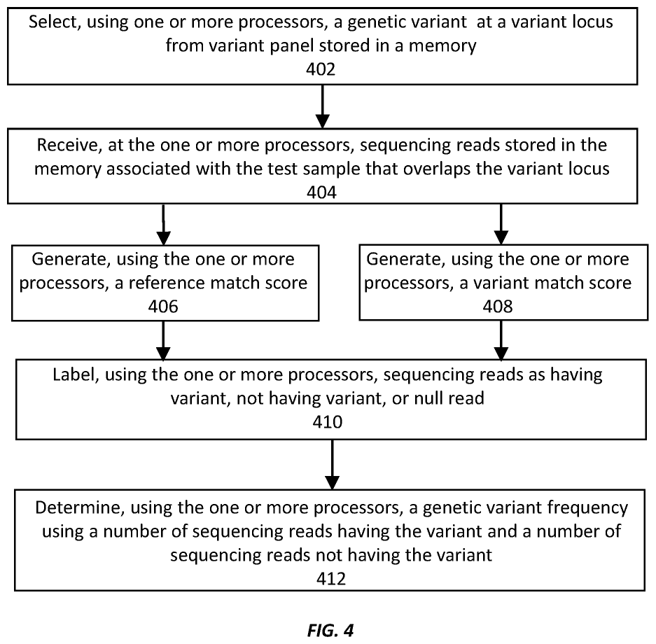

FIG. 4 shows an exemplary computer-implemented method for determining a variant frequency in a test sample from a subject. Step 402 includes selecting, using one or more processors, a genetic variant at a variant locus from a variant panel stored in a memory. In some embodiments, this step includes receiving genetic variant and variant locus information for one or more variants from the variant panel stored in the memory. For example, the processor may accesses the memory to retrieve the genetic variant and variant locus information, which can be listed in a table or file stored on the memory. Selection is made from the variant panel through any suitable process (e.g., randomly, sequentially, using a prioritization rank). In some embodiments, the computer-implemented method is repeated until a desired number (or all) of the variants in the variant panel are analyzed.

Step 404 includes receiving, at the one or more processors, one or more sequencing reads stored in the memory, wherein the sequencing reads are associated with the test sample that overlaps the variant locus. For example, the processor may access the memory to retrieve the one or more sequencing reads that overlap the variant locus. The memory may store a table or file containing sequencing reads (e.g., a BAM or SAM file), which includes the read and the read locus. Those sequencing reads in the table or file that overlap with the locus of the selected variant can then be selected and received at the one or more processors.

Step 406 includes generating, using the one or more processors, a reference match score for each of the one or more sequencing reads by aligning each sequencing read to a corresponding reference sequence retrieved from the memory, wherein the corresponding reference sequence does not comprise the genetic variant. In some embodiments, this step includes receiving a reference sequence corresponding to the selected variant (i.e., a corresponding reference sequence). For example, the corresponding reference sequence may be stored in a table or file in the memory. In some embodiments, the table or file storing the corresponding reference sequence is the same table or file storing information about the selected variant or the variant panel. In some embodiments, the table or file storing the corresponding reference sequence is a different table or file from the table or file storing information about the selected variant or the variant panel. Each sequencing read corresponding to the selected variant and received at the one or more processors is aligned to the corresponding reference sequence using an alignment module. The alignment module implements an alignment algorithm (such as a Smith-Waterman alignment algorithm or a Needleman-Wunsch alignment algorithm) to generate the reference match score. In some embodiments, the reference match score is stored in the memory, for example by automatically updating the table or file storing the sequencing reads or by automatically generating a new table or file containing the reference match score and the associate read or a read identifier.

Step 408 includes generating, using the one or more processors, a variant match score for each of the one or more sequencing reads by aligning each sequencing read to a corresponding variant sequence retrieved from the memory, wherein the corresponding variant sequence comprises the genetic variant. In some embodiments, this step includes receiving a variant sequence corresponding to the selected variant (i.e., a corresponding variant sequence). For example, the corresponding variant sequence may be stored in a table or file in the memory (which may be the same file or table as the table or file storing the corresponding reference sequence, or a different file). In some embodiments, the table or file storing the corresponding variant sequence is the same table or file storing information about the selected variant or the variant panel. In some embodiments, the table or file storing the corresponding variant sequence is a different table or file from the table or file storing information about the selected variant or the variant panel. Each sequencing read corresponding to the selected variant and received at the one or more processors is aligned to the corresponding variant sequence using an alignment module. The alignment module implements an alignment algorithm (generally the same alignment algorithm used to align the sequencing read with the reference alignment module) to generate the variant match score. In some embodiments, the variant match score is stored in the memory, for example by automatically updating the table or file storing the sequencing reads or by automatically generating a new table or file containing the reference match score and the associate read or a read identifier. In some embodiments, a table or file is automatically generated that includes both the reference match score and the variant match score.

Step 410 includes labeling, using the one or more processors, each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal. In some embodiments, the step of labeling, using the one or more processors, each of the one or more sequencing reads as either having the genetic variant, not having the genetic variant, or a being a null read, is based on the reference match score and the variant match score is implemented by a labeling module. The labeling module can compare the variant match score and the reference match score. A sequencing read is labeled as having the genetic variant if the reference match score and the variant match score indicate that the sequencing read more closely matches the corresponding variant sequence than the corresponding reference sequence. The sequencing read is labeled as not having the genetic variant if the reference match score and the variant match score indicate that the sequencing read more closely matches the corresponding reference sequence than the corresponding variant sequence. Further, in some embodiments, the sequencing read is labeled as a null read if the reference match score and the variant match score are equal. In some embodiments, the label associated with the sequencing read is automatically stored in the memory. For example, in some embodiments, the one or more processors automatically accesses a table or file stored on the memory and updates the file to include the labels for the sequencing reads. In some embodiments, the one or more processors automatically generates a table or file and stores it on the memory, which includes the labels for the sequencing reads.

At step 412 includes determining, using the one or more processors, a genetic variant frequency using a number of sequencing reads having the variant and a number of sequencing reads not having the variant. In some embodiments, the one or more processors automatically generates or updates a table or file in the memory to record the genetic variant frequency.

The computer-implemented method for detecting a genetic variant or determining an allele frequency for the genetic variant in a test sample from a subject can include the use of an electronic system that includes one or more processors and a memory storing a reference sequence and a variant sequence pair. The reference sequence and the variant sequence pair correspond with a genetic variant being queried by the method, which may be selected, using the one or more processors, from a variant panel stored on the memory. The one or more processors can receive one or more sequencing reads from the test sample, wherein the sequencing reads overlap the genetic locus of the queried genetic variant. The one or more processors can also receive the reference sequence from the memory and generate a reference match score for each of the one or more sequencing reads by aligning each sequencing read to the corresponding reference sequence. Further, the one or more processors can receive the variant sequence from the memory and generate a variant match score for each of the one or more sequencing reads by aligning each sequencing rad to the corresponding variant sequence. Based on the reference match score and the variant match score, the sequencing reads can be labeled as having the genetic variant, not having the genetic variant, or being a null read. The sequencing read is labeled as having the genetic variant if the reference match score and the variant match score indicate that the sequencing read more closely matches the corresponding variant sequence than the corresponding reference sequence. The sequencing read is labeled as not having the genetic variant if the reference match score and the variant match score indicate that the sequencing read more closely matches the corresponding reference sequence than the corresponding variant sequence. Finally, the sequencing read is labeled as a null read if the reference match score and the variant match score are equal. The labeled sequencing reads may be stored in the memory, or a number of sequencing reads having the genetic variant and/or a number of sequencing reads not having the genetic variant (and, optionally, the number of null reads) may be stored in the memory. In some embodiments, the computer-implemented process can use the number of sequencing reads labeled as having the genetic variant and/or the number of sequencing reads labeled as not having the genetic variant to call the sample as having the variant and/or determine a variant allele frequency for the sample. This process may be repeated for any number of genetic variants to be queried.

In some embodiments, a computer-implemented method of detecting a genetic variant or determining an allele frequency for the genetic variant in a test sample from a subject, comprising, and an electronic device comprising one or more processors and a memory storing a reference sequence that does not comprise the genetic variant and a variant sequence comprising the genetic variant at a variant locus; receiving, at the one or more processors, one or more sequencing reads associated with the test sample that corresponds with the reference sequence and the variant sequence; receiving, at the one or more processors, the reference sequence from the memory; generating, at the one or more processors, a reference match score for each of the one or more sequencing reads by aligning each sequencing read to the corresponding reference sequence; receiving, at the one or more processors, the variant sequence from the memory; generating, at the one or more processors, a variant match score for each of the one or more sequencing reads by aligning each sequencing read to the corresponding variant sequence; and labeling, at the one or more processors, each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal. In some embodiments, the method further comprises storing a label associated with each sequencing read in the memory.

In some embodiments, the computer-implemented method may further include calling, using the one or more processors, the presence of the genetic variant in the test sample based on the labeled one or more sequencing reads. The call for the genetic variant can be stored, by the one or more processors, in the memory.

In some embodiments, the computer-implemented method may further include, using the one or more processors, determining a variant allele frequency of the genetic variant in the test sample based on the labeled one or more sequencing reads. The variant allele frequency call may be stored in the memory.

The computer-implemented method may rely on the use of a variant panel stored in the memory to generate the reference sequence and/or the variant sequence used according to the method. The method may include selecting, using the one or more processors, the genetic variant from the variant panel, generating, using the one or more processors, the reference sequence and/or the variant sequence; and storing the reference sequence and/or the variant sequence in the memory. In other embodiments, the reference sequence and or the variant sequenced used according to the method is pre-stored in the memory, and corresponds to the queried genetic variant.

In some embodiments, the computer-implemented method includes the automatic generation or updating of a report (such as an electronic medical record). The report can include one or more of a call for the presence or absence of the genetic variant, a call for the variant allele frequency, and/or a disease status. The report can also include identifying information for the subject (e.g., name, identification number, etc.). The report may be stored in the memory and/or transmitted to a second electronic device (for example, an electronic device of the subject or a healthcare provider of the subject).

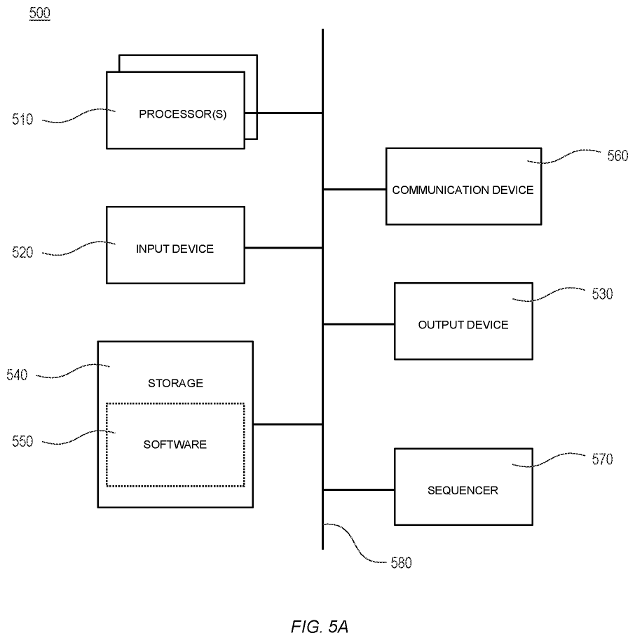

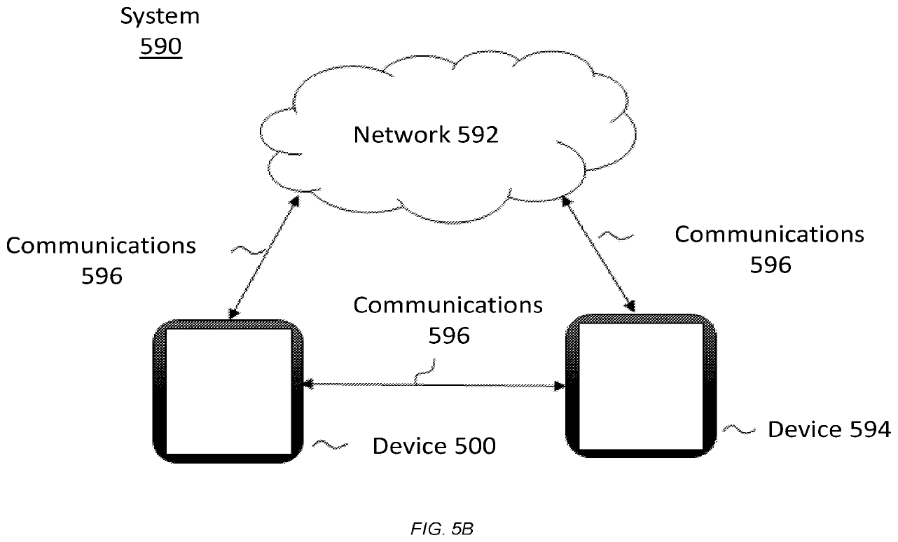

FIG. 5A shows an example of a computing device in accordance with one embodiment. Device 500 can be a host computer connected to a network. Device 500 can be a client computer or a server. As shown in FIG. 5A, device 500 can be any suitable type of microprocessor-based device, such as a personal computer, workstation, server or handheld computing device (portable electronic device) such as a phone or tablet. The device can include, for example, one or more of processor 510, input device 520, output device 530, storage 540, and communication device 560. Input device 520 and output device 530 can generally correspond to those described above, and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker. In some embodiments, the input device 520 and output device 530 can be the same or different devices.

Storage 540 can be any suitable device that provides storage, such as an electrical, magnetic or optical memory including a RANI (volatile and non-volatile), cache, hard drive, or removable storage disk. Communication device 560 can include any suitable device capable of transmitting and receiving signals over a network, such as a network interface chip or device. The components of the computer can be connected in any suitable manner, such as via a physical bus 580 or wirelessly (e.g., Bluetooth®, Wi-Fi®, or any other wireless technology).

Software 550, which can be stored in storage 540 and executed by processor 510, can include, for example, the programming that embodies the functionality of the present disclosure (e.g., as embodied in the devices as described above).

Software 550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can implement any operating system suitable for operating on the network. Software 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 510.

Device 500 can further include a sequencer 570, which can be any suitable nucleic acid sequencing instrument.

FIG. 5B shows an example of a computing system in accordance with one embodiment. In computing system 590, device 500 (e.g., as described above and illustrated in FIG. 5A) is connected to network 592, which is also connected to device 594. In some embodiments, device 594 is a sequencer (e.g., a next-generation sequencer). Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 500 and 594 may communicate, e.g., using suitable communication interfaces via network 592, such as a Local Area Network (LAN), Virtual Private Network (VPN), or the Internet. In some embodiments, network 592 can be, for example, the Internet, an intranet, a virtual private network, a cloud network, a wired network, or a wireless network. Devices 500 and 594 may communicate, in part or in whole, via wireless or hardwired communications, such as Ethernet, IEEE 802.11b wireless, or the like. Additionally, devices 500 and 594 may communicate, e.g., using suitable communication interfaces, via a second network, such as a mobile/cellular network. Communication between devices 500 and 594 may further include or communicate with various servers such as a mail server, mobile server, media server, telephone server, and the like. In some embodiments, Devices 500 and 594 can communicate directly (instead of, or in addition to, communicating via network 592), e.g., via wireless or hardwired communications, such as Ethernet, IEEE 802.11b wireless, or the like. In some embodiments, devices 500 and 594 communicate via communications 596, which can be a direct connection or can occur via a network (e.g., network 592).

One or all of devices 500 and 594 generally include logic (e.g., http web server logic) or are programmed to format data, accessed from local or remote databases or other sources of data and content, for providing and/or receiving information via network 592 according to various examples described herein.

In an exemplary embodiment, there is an electronic device comprising: one or more processors; a memory; and one or more programs, wherein the one or more programs are stored in the memory and configured to be executed by the one or more processors, the one or more programs including instructions for: (a) selecting a genetic variant at a variant locus from a variant panel; (b) obtaining one or more sequencing reads associated with a test sample that overlaps the variant locus; (c) generating a reference match score for each of the one or more sequencing reads by aligning each sequencing read to a corresponding reference sequence, wherein the corresponding reference sequence does not comprise the genetic variant; (d) generating a variant match score for each of the one or more sequencing reads by aligning each sequencing read to a corresponding variant sequence, wherein the corresponding variant sequence comprises the genetic variant; and (e) labeling each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

In another exemplary embodiment, there is a non-transitory computer-readable storage medium storing one or more programs, the one or more programs comprising instructions, which when executed by one or more processors of an electronic device having a display, cause the electronic device to: (a) select a genetic variant at a variant locus from a variant panel; (b) obtain one or more sequencing reads associated with the test sample that overlaps the variant locus; (c) generate a reference match score for each of the one or more sequencing reads by aligning each sequencing read to a corresponding reference sequence, wherein the corresponding reference sequence does not comprise the genetic variant; (d) generate a variant match score for each of the one or more sequencing reads by aligning each sequencing read to a corresponding variant sequence, wherein the corresponding variant sequence comprises the genetic variant; and (e) label each of the one or more sequencing reads as either having the genetic variant, not having the genetic variant, or a being a null read, based on the reference match score and the variant match score; wherein: a sequencing read is labeled as having the genetic variant if the reference match score and the variant match score indicate that the sequencing read more closely matches the corresponding variant sequence than the corresponding reference sequence; a sequencing read is labeled as not having the genetic variant if the reference match score and the variant match score indicate that the sequencing read more closely matches the corresponding reference sequence than the corresponding variant sequence; and a sequencing read is labeled as a null read if the reference match score and the variant match score are equal.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

### EXAMPLES

The examples provided herein are included for illustrative purposes only and are not intended to limit the scope of the invention.

### Example 1

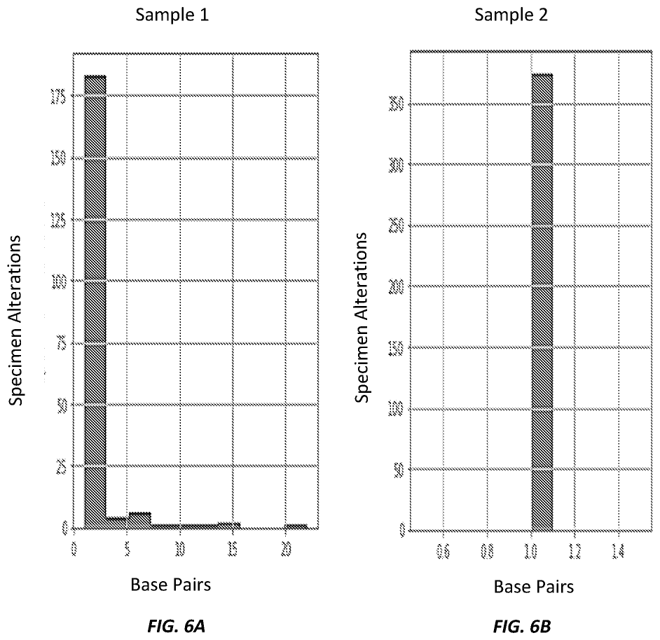

Sequencing reads from Sample 1 and Sample 2 were initially obtained using targeted sequencing methods and variants and allele depths called using standard variant calling protocols to generate curated sets of variants from the baseline sample. Variant panels and allele depths were selected for Sample 1 and Sample 2. Variants in the variant panel for Sample 1 ranged from 1 to 22 bases in length (FIG. 6A), and variants in in the variant panel for Sample 2 included only variants of a single base length (FIG. 6B).

Reference sequences corresponding to each variant in the variant panel (i.e., a corresponding reference sequence) and a variant sequence corresponding to each variant in the variant panel (i.e., a variant reference sequence) were generated. The variant or reference base(s) were flanked with 200 bases on each side of the variant locus to generate the corresponding variant sequence and the corresponding reference sequence.

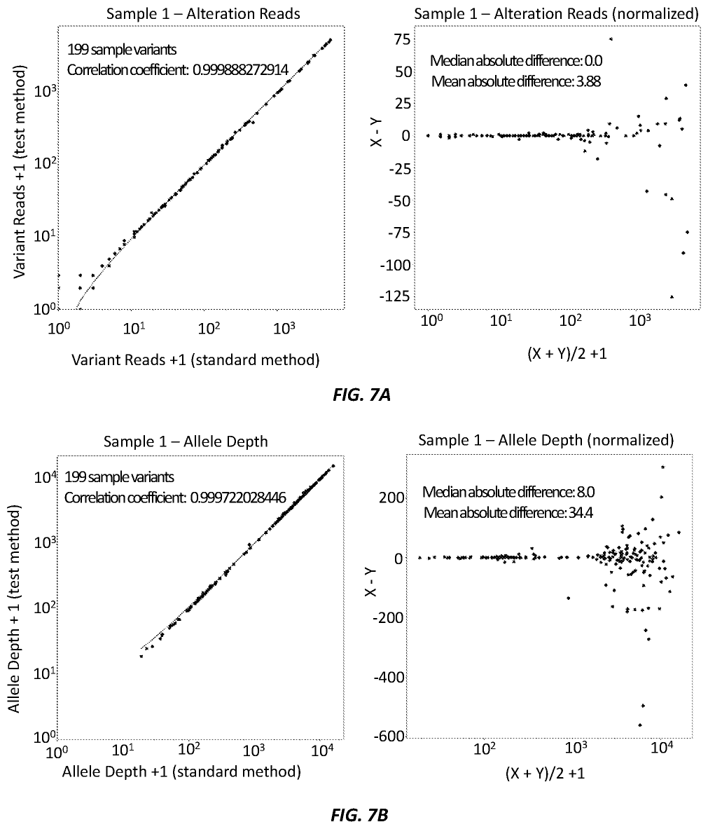

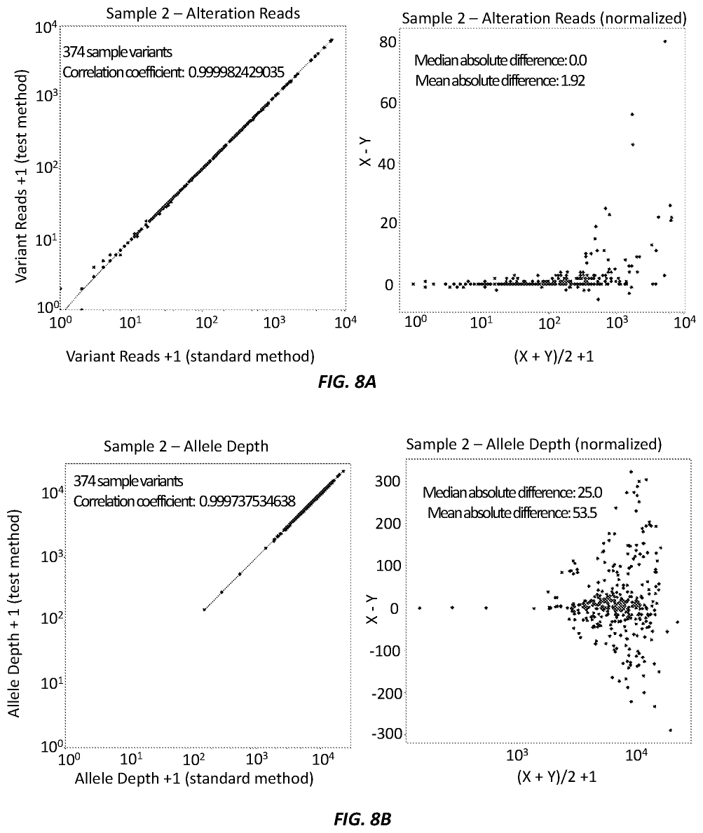

Each sequencing read from Sample 1 and Sample 2 that overlapped a variant locus of a variant in the variant panel was aligned with a corresponding reference sequence and a corresponding variant sequence using a Striped Smith-Waterman alignment algorithm to generate a reference match score and a variant match score, respectively. Using the match scores, the reads were labeled as either having the variant, not having the variant, or a null read. 199 variants from Sample 1 were detected, and 374 variants from Sample 2 were detected. FIG. 7A and FIG. 8A show a plot of the number of variant reads detected by comparing the match scores (y-axis) against the number of variant reads detected using the standard variant calling protocol (x-axis) in log scale (left) and normalized (right) for Sample 1 (FIG. 7A) and Sample 2 (FIG. 8A). FIG. 7B and FIG. 8B show a plot of the variant allele depth at each variant locus for the sum of sequencing reads labeled as having the variant or not having the variant (i.e., excluding null reads) (y-axis) against the variant locus depth at each variant locus for the sum of sequencing reads from the initial pool of sequencing reads that overlap with the variant locus (x-axis) in log scale (left) and normalized (right) for Sample 1 (FIG. 7B) and Sample 2 (FIG. 8B).

### Example 2

Sequencing reads from Sample 1 and Sample 2 were initially obtained using targeted sequencing methods and variants and allele depths called using standard variant calling protocols to generate curated sets of variants from the baseline sample. Variant panels and allele depths were selected for Sample 1 and Sample 2. Variants in the variant panel for Sample 1 ranged from 1 to 22 bases in length (FIG. 6A), and variants in in the variant panel for Sample 2 included only variants of a single base length (FIG. 6B).

Reference sequences corresponding to each variant in the variant panel (i.e., a corresponding reference sequence) and a variant sequence corresponding to each variant in the variant panel (i.e., a variant reference sequence) were generated. The variant or reference base(s) were flanked with 500 bases on each side of the variant locus to generate the corresponding variant sequence and the corresponding reference sequence.

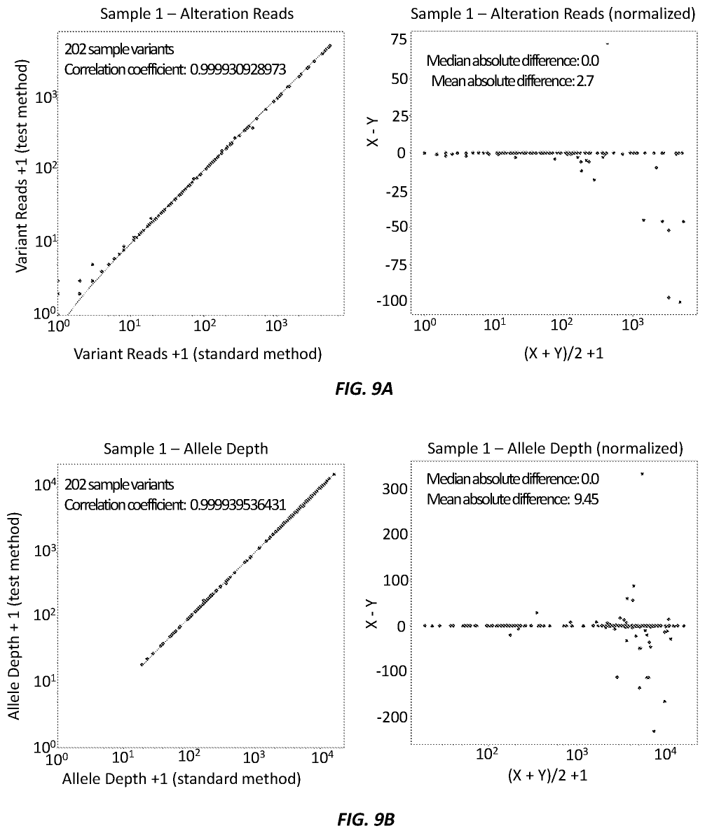

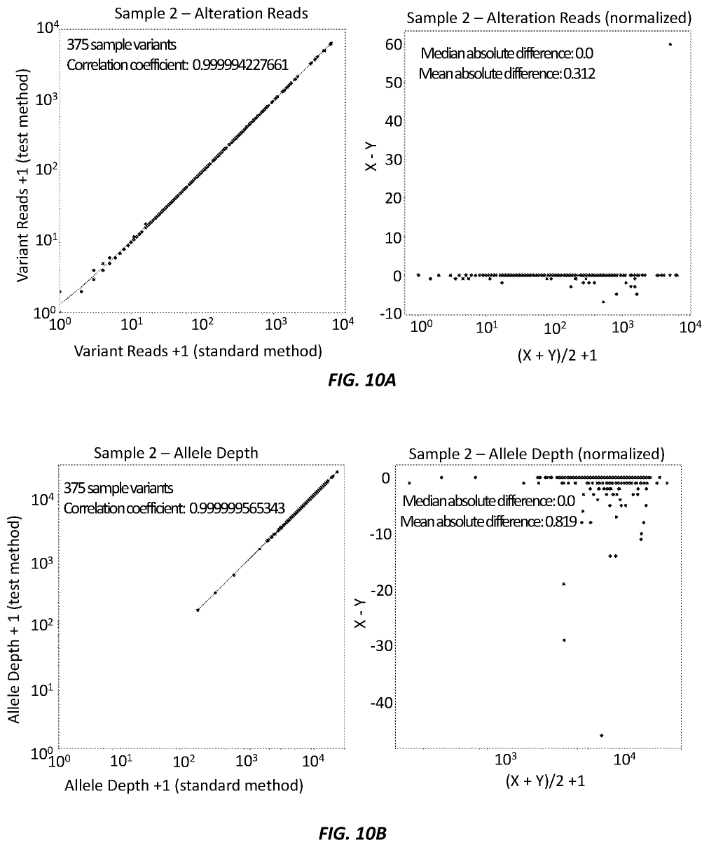

Each sequencing read from Sample 1 and Sample 2 that overlapped a single base of a variant locus of a variant in the variant panel was aligned with a corresponding reference sequence and a corresponding variant sequence using a Striped Smith-Waterman alignment algorithm to generate a reference match score and a variant match score, respectively. Using the match scores, the reads were labeled as either having the variant, not having the variant, or a null read. 202 variants from Sample 1 were detected, and 375 variants from Sample 2 were detected. FIG. 9A and FIG. 10A show a plot of the number of variant reads detected by comparing the match scores (y-axis) against the number of variant reads detected using the standard variant calling protocol (x-axis) in log scale (left) and normalized (right) for Sample 1 (FIG. 9A) and Sample 2 (FIG. 10A). FIG. 9B and FIG. 10B show a plot of the variant locus depth at each variant locus for the sum of sequencing reads labeled as having the variant or not having the variant (i.e., excluding null reads) (y-axis) against the variant locus depth at each variant locus for the sum of sequencing reads from the initial pool of sequencing reads that overlap with the variant locus (x-axis) in log scale (left) and normalized (right) for Sample 1 (FIG. 9B) and Sample 2 (FIG. 10B).

1. A method of detecting a genetic variant or determining a variant allele frequency in a test sample from a subject, comprising:
receiving, at one or more processors, one or more sequencing reads that corresponds with a reference sequence and a variant sequence of the test sample;
receiving, at the one or more processors, the reference sequence from a memory;
generating, at the one or more processors, a reference match score for each of the one or more sequencing reads by aligning each sequencing read to the corresponding reference sequence;
receiving, at the one or more processors, the variant sequence from the memory;
generating, at the one or more processors, a variant match score for each of the one or more sequencing reads by aligning each sequencing read to the corresponding variant sequence; and
labeling, at the one or more processors, each of the one or more sequencing reads as (1) having the genetic variant, (2) not having the genetic variant, or (3) being a null read, based on the reference match score and the variant match score; wherein:
a sequencing read is labeled as having the genetic variant if the reference match score and the variant match score indicate that the sequencing read more closely matches the corresponding variant sequence than the corresponding reference sequence;
a sequencing read is labeled as not having the genetic variant if the reference match score and the variant match score indicate that the sequencing read more closely matches the corresponding reference sequence than the corresponding variant sequence; and
a sequencing read is labeled as a null read if the reference match score and the variant match score are equal.

2-8. (canceled)

9. A method comprising:
receiving, at one or more processors, one or more sequencing reads associated with a test sample that corresponds with a reference sequence and a variant sequence;
receiving, at the one or more processors, the reference sequence;
generating, at the one or more processors, a reference match score for each of the one or more sequencing reads by aligning each sequencing read to the corresponding reference sequence;
receiving, at the one or more processors, the variant sequence;
generating, at the one or more processors, a variant match score for each of the one or more sequencing reads by aligning each sequencing read to the corresponding variant sequence; and
labeling, at the one or more processors, each of the one or more sequencing reads as (1) having the genetic variant, (2) not having the genetic variant, or (3) being a null read, based on the reference match score and the variant match score; wherein:
a sequencing read is labeled as having the genetic variant if the reference match score and the variant match score indicate that the sequencing read more closely matches the corresponding variant sequence than the corresponding reference sequence;
a sequencing read is labeled as not having the genetic variant if the reference match score and the variant match score indicate that the sequencing read more closely matches the corresponding reference sequence than the corresponding variant sequence; and
a sequencing read is labeled as a null read if the reference match score and the variant match score are equal.

10. The method of claim 9, comprising storing a label associated with each sequencing read labeled as having the genetic variant and/or each sequencing read labeled as not having the variant in the memory.

11. The method of claim 9, further comprising calling, using the one or more processors, a presence or absence of the genetic variant in the test sample based on the labeled one or more sequencing reads; and storing a call for the genetic variant in the memory.

12. The method of claim 9, further comprising determining, using the one or more processors, the variant allele frequency of the genetic variant in the test sample based on the labeled one or more sequencing reads; and storing the variant allele frequency in the memory.

13. The method of claim 9, comprising, using the one or more processors:
selecting, using the one or more processors, the genetic variant from a variant panel stored on the memory;
generating, using the one or more processors, the reference sequence or the variant sequence; and
storing the reference sequence or the variant sequence in the memory.

14. The method of claim 9, wherein the one or more sequencing reads comprises a plurality of sequencing reads overlapping the variant locus, the method further comprising determining, using the one or more processors, a number of sequencing reads from the plurality of sequencing reads having the genetic variant or a number of sequencing reads from the plurality of sequencing reads not having the genetic variant.

15. The method of claim 9, comprising labeling, using the one or more processors, one or more sequencing reads associated with the test sample for a plurality of genetic variants at different variant loci selected from a variant panel.

16. The method of claim 9, comprising determining, using the one or more processors, a disease status for the subject.

17. The method of claim 9, comprising generating, using the one or more processors, a report comprising (1) identifying information for the subject, and (2) a call for the presence or absence of the genetic variant, or a call for the variant allele frequency.

18. The method of claim 17, comprising transmitting the report to a second electronic device.

19. (canceled)

20. A method of detecting a genetic variant or determining a variant allele frequency in a test sample from a subject, comprising:
selecting the genetic variant at a variant locus from a variant panel;
obtaining one or more sequencing reads associated with the test sample that overlaps the variant locus;
generating a reference match score for each of the one or more sequencing reads by aligning each sequencing read to a corresponding reference sequence, wherein the corresponding reference sequence does not comprise the genetic variant;
generating a variant match score for each of the one or more sequencing reads by aligning each sequencing read to a corresponding variant sequence, wherein the corresponding variant sequence comprises the genetic variant; and
labeling each of the one or more sequencing reads as (1) having the genetic variant, (2) not having the genetic variant, or (3) being a null read, to generate labeled sequencing reads, based on the reference match score and the variant match score; wherein:
a sequencing read is labeled as having the genetic variant if the reference match score and the variant match score indicate that the sequencing read more closely matches the corresponding variant sequence than the corresponding reference sequence;
a sequencing read is labeled as not having the genetic variant if the reference match score and the variant match score indicate that the sequencing read more closely matches the corresponding reference sequence than the corresponding variant sequence; and
a sequencing read is labeled as a null read if the reference match score and the variant match score are equal.

21-30. (canceled)

31. The method of claim 16, wherein the disease status is a value proportional to a percentage of circulating-tumor DNA (ctDNA) compared to total cell-free DNA (cfDNA) in the test sample.

32. The method of claim 16, wherein the disease status is a maximum somatic allele fraction of cfDNA.

33. The method of claim 16, wherein the disease status comprises a qualitative factor indicating recurrence of a cancer in the subject, the presence of a cancer resistant to a treatment modality in the subject, or the presence of a cancer that can be treated with a particular treatment modality.

34. The method of claim 9, wherein the reference match score and the variant match score are determined using a sequence alignment algorithm.

35-40. (canceled)

41. The method of claim 9, comprising generating the one or more sequencing reads by sequencing nucleic acid molecules in the test sample.

42-58. (canceled)

59. A method of monitoring disease progression or recurrence, comprising:
sequencing nucleic acid molecules in a first test sample acquired from a subject with a disease to generate first sequencing reads;
generating a personalized variant panel for the subject;
sequencing nucleic acid molecules in a second test sample acquired from the subject at a later time point than the first test sample to generate second sequencing reads; and
detecting, using the second sequencing reads, the genetic variant or determining, using the second sequencing read, the variant allele frequency according to the method of claim 9.

60. The method of claim 59, comprising administering a disease therapy to the subject after the first test sample is acquired from the subject and before the second test sample is acquired from the subject.

61-67. (canceled)

68. A method of treating a subject with a disease, comprising:
acquiring a first test sample from the subject;
sequencing nucleic acid molecules in a first test sample to generate first sequencing reads;
determining a first disease status using the first sequencing reads;
generating a personalized variant panel for the subject;
administering a disease therapy to the subject;
acquiring a second test sample from the subject after the disease therapy has been administered to the subject;
sequencing nucleic acid molecules in the second test sample to generate second sequencing reads;
detecting, using the second sequencing reads, the genetic variant or determining, using the second sequencing read, the variant allele frequency according to the method of claim 9;
determining a second disease status based on the second sequencing reads;
determining disease progression by comparing the first disease status and the second disease status;
adjusting the disease therapy administered to subject based on the disease progression; and
administering the adjusted disease therapy to the subject.

69-150. (canceled)
